(19) United States
(12) Patent Application Publication
(10) Pub. No.: US 2005/0106249 A1
(43) Pub. Date: May 19, 2005

(54) Once-a-day, oral, controlled-release, oxycodone dosage forms

(76) Inventors: Stephen Hwang, Palo Alto, CA (US); Nishit B. Modi, Sunnyvale, CA (US); Padmaja Shivanand, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/977,403

(22) Filed: Oct. 28, 2004

Related U.S. Application Data
(63) Continuation-in-part of application No. 10/447,910, filed on May 28, 2003.
(63) Continuation-in-part of application No. 10/423,454, filed on Apr. 25, 2003.
(63) Continuation-in-part of application No. 10/447,910, filed on May 28, 2003.
(60) Provisional application No. 60/515,880, filed on Oct. 29, 2003.
(60) Provisional application No. 60/376,470, filed on Apr. 29, 2002.
(60) Provisional application No. 60/384,442, filed on May 31, 2002.

(51) Int. Cl. A61K 9/26

(52) U.S. Cl. 424/469

(57) ABSTRACT

Oxycodone formulations are provided which produce substantially flat in vivo steady state plasma profiles. Tolerance levels associated with such profiles and tolerance levels associated with biphasic profiles are shown not to be statistically different. The substantially flat in vivo steady state plasma profiles are produced by dosage forms having substantially zero order in vitro release profiles. Such release profiles produce low single dose in vivo Cₘₐₓ levels which can reduce the probability of adverse side effects.

### I. CROSS REFERENCE TO RELATED APPLICATIONS

This application claims the benefit under 35 USC §119(e) of U.S. Provisional Application No. 60/515,880 filed Oct. 29, 2003, the contents of which in its entirety is hereby incorporated by reference.

This application is a Continuation-In-Part of U.S. application Ser. No. 10/423,454 filed Apr. 25, 2003, which claims the benefit under 35 USC §119(e) of U.S. Provisional Application No. 60/376,470 filed Apr. 29, 2002, and which was published as U.S. Patent Publication No. 2004-0010000 A1 on Jan. 15, 2004 and as WO 03/092648 on Nov. 13, 2003, the contents of all of which in their entireties are hereby incorporated by reference.

This application is also a Continuation-In-Part of U.S. application Ser. No. 10/447,910 filed May 28, 2003, which claims the benefit under 35 USC §119(e) of U.S. Provisional Application No. 60/384,442 filed May 31, 2002, and which was published as U.S. patent Publication No. 2003-0224051 A1 on Dec. 4, 2003, and as WO 03/101384 on Dec. 11, 2003, the contents of all of which in their entireties are hereby incorporated by reference.

### II. FIELD OF THE INVENTION

This invention relates to in vitro and in vivo profiles, i.e., in vitro dissolution/release profiles and in vivo single dose and in vivo steady state plasma profiles, for the opioid analgesic, oxycodone, when administered orally using a controlled-release dosage form. In particular, the invention relates to in vitro and in vivo oxycodone profiles designed to produce effective pain management and a reduced probability of “liking” when oxycodone is orally administered to a patient on a once-a-day basis.

### III. BACKGROUND OF THE INVENTION

A. Oxycodone

Oxycodone, a Schedule II drug, is an opioid for the management of moderate to severe chronic pain, such as, pain due to surgery, cancer, trauma, biliary colic, renal colic, myocardial infarction and burns. Oxycodone has been marketed as an analgesic for more than 70 years. It is currently available in immediate release (IR) forms, as well as in a controlled release (CR) formulation indicated for b.i.d. dosing.

The pharmacological and medical properties of analgesic opioids including oxycodone are known in Pharmaceutical Sciences, Remington, 17th Ed., pp. 1099-1107 (1985), and The Pharmacological Basis of Therapeutics, Goodman and Rall, 8th Ed., pp. 485-518 (1990). Generally, the analgesic action of parenterally administered oxycodone is apparent within 15 minutes, while the onset of action of orally administered oxycodone is somewhat slower with analgesia occurring within about 30 minutes. In human plasma, the half-life of orally administered immediate release oxycodone is about 3.2 hours. Physicians' Desk Reference, Thompson Healthcare, 56^{th} Ed., pp. 2912-2918 (2002).

In the past, oxycodone has been administered in conventional forms, such as nonrate-controlling, dose-dumping immediate release tablets, or by dose-dumping capsules, and usually at multiple, repetitive dosing intervals throughout the day. Oxycodone is also administered on a twice-a-day basis with a controlled release matrix system, OXYCONTIN® (Purdue Pharma LP, Stamford, Conn.). The OXYCONTIN® mode of therapy, however, continues to lead to an initial high dose of oxycodone in the blood after administration, followed by decreased levels of oxycodone in the blood. Moreover, this peak and trough pattern occurs twice during a 24-hour period due to the twice-a-day dosing regimen. The concentration differences in dosing patterns are related to the presence and absence of administered drug, which is a major disadvantage associated with these prior dosage forms. Conventional dosage forms and their mode of operation, including dose peaks and valleys, are discussed in Pharmaceutical Sciences, Remington, 18th Ed., pp. 1676-1686 (1990), Mack Publishing Co.

B. Tolerance to Oxycodone

Previous studies in rats and mice with opioids have shown the development of tolerance to analgesia (antinociception) after bolus dosing, intermittent dosing, and constant rate infusions (Ekblom et al 1993, Gardmark et al 1993, Ouellet & Pollack 1995, 1997, Duttaroy & Yoburn 1995).

In connection with the OXYCONTIN® product, employees of Purdue Pharma and its associated companies have published scientific articles in which biphasic profiles are described as being better than flat profiles with regard to the development of oxycodone tolerance. Thus, in the Journal of Pain and Symptom Management, Purdue employees wrote (Benziger et al. 1997 at page 81):

“Although the benefits of controlled-release dosage forms that permit less frequent dosing are well established, it has been suggested that the maintenance of nearly constant plasma concentrations of opioids may lead to tolerance development. The CR oxycodone tablets under study [OXYCONTIN®] were developed to reduce the number of Cₘᵢₙ/Cₘₐₓ fluctuations during the 12-hr dosing interval while matching the degree of fluctuation (Cₘᵢₙ/Cₘₐₓ) in plasma oxycodone concentrations observed during steady-state dosing with comparable doses of IR oxycodone. By retaining the degree of fluctuation in plasma concentrations the possibility of diminished pharmacodynamic effects over time may be diminished as compared to CR formulations that maintain comparable constant blood levels.” (citations omitted.)

Similarly, Dr. Robert Kaiko, an inventor of OXYCONTIN®, wrote in Acta Annesthesiol Scand (Kaiko 1997 at page 172):

“Another rationale basis for the biphasic opioid absorption profile is to produce a peak-to-trough fluctuation comparable to the conventional immediate-release opioid. Because it has been suggested that very steady plasma opioid concentrations may lead to tolerance development, it is anticipated that alteration of the rate of fluctuation without alteration of the degree of fluctuation would minimize tolerance development.” (citations omitted.)

Further teachings against flat plasma profiles and, in particular, against flat plasma profiles for once-a-day dosage forms, can be found in the patent literature. Thus, U.S. Pat. No. 5,478,577, assigned to Euroceltique, S. A., a company related to Purdue Pharma, states (column 5, lines 34-42):

“It has now been surprisingly discovered that quicker and greater analgesic efficacy is achieved by 24 hour oral opioid formulations which do not exhibit a substantially flat serum concentration curve, but which instead provide a more rapid initial opioid release so that the minimum effective analgesic concentration can be more quickly approached in many patients who have measurable if not significant pain at the time of dosing.”

See also Euroceltique's U.S. Pat. No.5,672,360, column 5, lines 40-47.

In view of these explicit warnings against flat profiles by the leading manufacturer of controlled-release oxycodone products, persons of ordinary skill in the art have been led away from the use of oxycodone dosage forms having substantially zero order in vitro release profiles. In particular, such persons would expect that flat profiles would generate higher levels of tolerance than biphasic profiles.

As discussed fully below (see Example 8), it has been found that notwithstanding Purdue Pharma's teachings, oxycodone tolerance levels associated with biphasic profiles and flat profiles (substantially zero order release profiles) are, in fact, not statistically different. In addition, as illustrated in FIG. 5 (see discussion below), substantially zero order oxycodone release profiles produce low single dose Cₘₐₓ values and thus are expected to have lower levels of “liking” than profiles that are not substantially zero order, such as biphasic profiles. As has been well-documented in the literature, including the popular press, Purdue Pharma's biphasic OXYCONTIN® product has serious abuse problems, substantially beyond any issue of “liking.”

Importantly, as illustrated by the efficacy data of Example 7 below, substantially zero order oxycodone release profiles achieve effective pain management. Accordingly, in accordance with the invention, it has been shown that oxycodone dosage forms which have substantially zero order in vitro release profiles can be used to achieve effective pain management without exaggerated tolerance problems and with a reduced probability of “liking”—a combination of benefits not previously known or expected from the existing state of the art.

### IV. SUMMARY OF THE INVENTION

In accordance with a first aspect, the invention provides a controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said formulation providing (a) a mean, single dose, maximum plasma concentration Cₘₐₓ and (b) a mean, single dose, area under a plasma concentration-time curve for 0-48 hours AUC₀₋₄₈ which satisfy the relationships:
   3.5×10⁻⁴ liter⁻¹≦Cₘₐₓ/D≦6.8×10⁻⁴ liter⁻¹, and
   7.6×10⁻³ hour/liter≦AUC₀₋₄₈/D≦16.7×10⁻³ hour/liter,
   wherein said formulation provides pain relief for about 24 hours or more after administration to the patient.

In accordance with a second aspect, the invention provides a controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   wherein:
(a) the formulation provides a mean, single dose, plasma concentration profile that increases substantially monotonically over 24 hours or more;
(b) the formulation provides a mean, single dose, area under a plasma concentration-time curve for 0-48 hours AUC₀₋₄₈ which satisfies the relationship:
   7.6×10⁻³ hour/liter≦AUC₀₋₄₈/D≦16.7×10⁻³ hour/liter; and
(c) the formulation provides pain relief for about 24 hours or more after administration to the patient.

In accordance with a third aspect, the invention provides a controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said formulation providing (a) a mean, single dose, 12 hour plasma concentration C₁₂ and (b) a mean, single dose, area under a plasma concentration-time curve for 0-48 hours AUC₀₋₄₈ which satisfy the relationships:
   2.7×10⁻⁴ liter⁻¹<C₁₂/D≦5.7×10⁻⁴ liter⁻¹, and
   7.6×10⁻³ hour/liter≦AUC₀₋₄₈/D≦16.7×10⁻³ hour/liter,
   wherein said formulation provides pain relief for about 24 hours or more after administration to the patient.

In accordance with a fourth aspect, the invention provides a controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said formulation providing mean, steady state, areas under a plasma concentration-time curve for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ which satisfy the relationships:
   AUC₀₋₆/AUC₀₋₂₄>0.18,
   AUC₆₋₁₂/AUC₀₋₂₄>0.18,
   AUC₁₂₋₁₈/AUC₀₋₂₄>0.18, and
   AUC₁₈₋₂₄/AUC₀₋₂₄>0.18,
   wherein said formulation provides pain relief for about 24 hours or more after administration to the patient.

In accordance with a fifth aspect, the invention provides a controlled-release oxycodone formulation for once-a-day oral administration to human patients comprising a dose of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said formulation having an in vitro release profile in which:
(a) 0-20% of the dose is released in 0-2 hours;
(b) 30-65% of the dose is released in 0-12 hours; and
(c) 80-100% of the dose is released in 0-24 hours;
   wherein the release profile is determined using a USP Type VII bath indexer in a constant temperature water bath at 37° C. and wherein said formulation provides pain relief for about 24 hours or more after administration to the patient.

In accordance with a sixth aspect, the invention provides a controlled-release oxycodone formulation for once-a-day oral administration to human patients comprising a dose of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   wherein:
(a) the dose comprises a first component for immediate release and a second component for sustained release; and
(b) the weight ratio W of the first component to the sum of the first and second components is less than about 0.25.

In accordance with a seventh aspect, the invention provides a method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said dosage form providing (a) a mean, single dose, maximum plasma concentration Cₘₐₓ and (b) a mean, single dose, area under a plasma concentration-time curve for 0-48 hours AUC₀₋₄₈ which satisfy the relationships:
   3.5×10⁻⁴ liter⁻¹≦Cₘₐₓ/D≦6.8×10⁻⁴ liter⁻¹, and
   7.6×10⁻³ hour/liter≦AUC₀₋₄₈/D≦16.7×10⁻³ hour/liter,
   wherein the dosage form provides pain relief for about 24 hours or more after administration to the patient.

In accordance with an eight aspect, the invention provides a method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   wherein:
(a) the dosage form provides a mean, single dose, plasma concentration profile that increases substantially monotonically over 24 hours or more;
(b) the dosage form provides a mean, single dose, area under a plasma concentration-time curve for 0-48 hours AUC₀₋₄₈ which satisfies the relationship:
   7.6×10⁻³ hour/liter≦AUC₀₋₄₈/D≦16.7×10⁻³ hour/liter; and
(c) the dosage form provides pain relief for about 24 hours or more after administration to the patient.

In accordance with a ninth aspect, the invention provides a method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said dosage form providing (a) a mean, single dose, 12 hour plasma concentration C₁₂ and (b) a mean, single dose, area under a plasma concentration-time curve for 0-48 hours AUC₀₋₄₈ which satisfy the relationships:
   2.7×10⁻⁴ liter⁻¹≦C₁₂/D≦5.7×10⁻⁴ liter⁻¹, and
   7.6×10⁻³ hour/liter≦AUC₀₋₄₈/D≦16.7×10⁻³ hour/liter,
   wherein said dosage form provides pain relief for about 24 hours or more after administration to the patient.

In accordance with a tenth aspect, the invention provides a method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said dosage form providing mean, steady state, areas under a plasma concentration-time curve for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ which satisfy the relationships:
   AUC₀₋₆/AUC₀₋₂₄>0.18,
   AUC₆₋₁₂/AUC₀₋₂₄>0.18,
   AUC₁₂₋₁₈/AUC₀₋₂₄>0.18, and
   AUC₁₈₋₂₄/AUC₀₋₂₄>0.18,
   wherein said dosage form provides pain relief for about 24 hours or more after administration to the patient.

In accordance with an eleventh aspect, the invention provides a method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   said dosage form providing pain relief for about 24 hours or more after administration to the patient and having an in vitro release profile in which:
(a) 0-20% of the dose is released in 0-2 hours;
(b) 30-65% of the dose is released in 0-12 hours; and
(c) 80-100% of the dose is released in 0-24 hours;
   where the release profile is determined using a USP Type VII bath indexer in a constant temperature water bath at 37° C.

In accordance with a twelfth aspect, the invention provides a method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
   wherein:
(a) the dose comprises a first component for immediate release and a second component for sustained release;
(b) the weight ratio W of the first component to the sum of the first and second components is less than about 0.25; and
(c) the dosage form provides pain relief for about 24 hours or more after administration to the patient.

The various AUC and C values referred to above can be determined using plasma samples from individuals to whom one or more opioid antagonists (e.g., naltrexone) have been administered or by using samples from individuals to whom an antagonist has not been administered. For higher dosage levels, antagonists are normally used, especially in studies involving healthy volunteers. For example, various of the numerical values set forth above are based on the pharmacokinetic data of Example 6, which used healthy volunteers and a dosage form which contained 80 mg of oxycodone HCl. As described in Example 6, naltrexone was administered in this study. As known in the art, naltrexone has a tendency to increase plasma oxycodone concentrations. Accordingly, somewhat lower AUC and C values would be expected if naltrexone had not been used, but the changes would not be expected to move the mean values substantially outside of the ranges specified.

Additional features and advantages of the invention are set forth in the detailed description which follows, and in part will be readily apparent to those skilled in the art from that description or recognized by practicing the invention as described herein. It is to be understood that both the foregoing general description and the following detailed description are merely exemplary of the invention, and are intended to provide an overview or framework for understanding the nature and character of the invention as it is claimed. Also, the above listed aspects of the invention, as well as the preferred and other embodiments of the invention discussed below, can be used separately or in any and all combinations.

The accompanying drawings are included to provide a further understanding of the invention, and are incorporated in and constitute a part of this specification. The drawings illustrate various embodiments of the invention, and together with the description serve to explain the principles and operation of the invention. The drawings and, in particular, FIGS. 1-4, are not intended to indicate scale or relative proportions of the elements shown therein. In the drawings and the specification, like parts in related figures are identified by like numbers.

### V. BRIEF DESCRIPTION OF THE DRAWINGS

FIG. 1 illustrates one type of dosage form that can be used in the practice of the invention. The dosage form is shown in FIG. 1 prior to administration to a subject.

FIG. 2 illustrates a first embodiment of the dosage form of FIG. 1 in opened section. As shown, the dosage form comprises an internally-housed, pharmaceutically-acceptable therapeutic oxycodone composition.

FIG. 3 illustrates a second embodiment of the dosage form of FIG. 1 in opened section. As shown, the dosage form comprises an internally-housed, pharmaceutically-acceptable therapeutic oxycodone composition and a separate and contacting displacement composition comprising means for pushing the pharmaceutical oxycodone composition from the dosage form.

FIG. 4 illustrates a dosage form which further includes an immediate-release overcoat of a pharmaceutically-acceptable therapeutic oxycodone composition.

FIG. 5 is a plot of simulated single dose plasma concentrations for a substantially zero order (SZO) release rate (curve 100), a fast-followed-by-slow release rate (curve 102), and a slow-followed-by-fast release rate (curve 104).

FIG. 6 is a plot of a preferred cumulative release range for the dosage forms of the invention. The vertical axis plots percent cumulative release of oxycodone and/or one or more of its pharmaceutically-acceptable acid addition salts (e.g., % of label claim for a dosage form that has received regulatory approval) and the horizontal axis plots time.

FIGS. 7A and 7B are plots of in vitro release profiles for the 17 mg oxycodone HCl dosage form identified as the “fast system” in Example 1. FIG. 7A (curve 106) plots the percent released per hour (e.g., % of label claim released per hour), while FIG. 7B (curve 108) plots the cumulative release in percent (e.g., cumulative % of label claim).

FIGS. 8A and 8B are plots of in vitro release profiles for the 17 mg oxycodone HCl dosage form identified as the “slow system” in Example 1. FIG. 8A (curve 110) plots the percent released per hour (e.g., % of label claim released per hour), while FIG. 8B (curve 112) plots the cumulative release in percent (e.g., cumulative % of label claim).

FIGS. 9A and 9B are plots of in vitro release profiles for the 20 mg oxycodone HCl dosage form of Example 2. FIG. 9A (curve 114) plots the percent released per hour (e.g., % of label claim released per hour), while FIG. 9B (curve 116) plots the cumulative release in percent (e.g., cumulative % of label claim).

FIGS. 10A and 10B are plots of in vitro release profiles for the 80 mg oxycodone HCl dosage form of Example 3. FIG. 10A (curve 118) plots the percent released per hour (e.g., % of label claim released per hour), while FIG. 10B (curve 120) plots the cumulative release in percent (e.g., cumulative % of label claim).

FIG. 11 is a plot of pupil diameter in millimeters (mm) versus time in hours for healthy male subjects who received placebo (curve 122), morphine (curve 124), or the dosage form of Example 2 (curve 126).

FIG. 12 is a plot of plasma concentrations in nanograms/milliliter (ng/mL) of oxycodone (curve 128), noroxycodone (curve 130), and oxymorphone (curve 132) versus time in hours for healthy male subjects who received the dosage form of Example 2.

FIG. 13 is a plot of simulated pharmacokinetics, specifically, single dose plasma concentrations, for immediate release (IR) dosing (q6h) (curve 134), as well as experimental data for the dosage form of Example 2 and a best-fit curve to that data (curve 136).

FIG. 14 is a plot of simulated pharmacokinetics, specifically, steady-state plasma concentrations, for immediate release (IR) dosing (q6h) (curve 140), OXYCONTIN biphasic dosing (curve 138), and substantially zero order/once-a-day (SZO-24) dosing using a dosage form having the overcoat/sustained release drug distribution of Example 2, i.e., 5% of the drug in the overcoat (curve 142). The y-axis in this figure shows oxycodone concentration.

FIGS. 15A and 15B are plots of mean in vivo plasma oxycodone concentration profiles for immediate release (IR) dosing (q6h) (curve 144; n=16), dosing with the 17 mg oxycodone HCl dosage form identified as the “fast system” in Example 1 (curve 146; n=17), and dosing with the 17 mg oxycodone HCl dosage form identified as the “slow system” in Example 1 (curve 148; n=17). FIG. 15A shows the single dose profiles and FIG. 15B shows the steady-state profiles. The error bars associated with the data points show the standard deviation (SD) in one direction.

FIGS. 16A, 16B, 16C, and 16D are plots of mean in vivo plasma oxycodone concentration profiles for substantially zero order (SZO) dosing with the 80 mg oxycodone HCl dosage form of Example 3 (curve 150; n=37), and biphasic dosing with 40 mg OXYCONTIN tablets (curve 152; n=38). FIG. 16A shows single dose and steady-state profiles, FIGS. 16B and 16C show single dose profiles, and FIG. 16D shows steady-state profiles. The error bars associated with the data points show the standard deviation (SD) in one direction.

FIG. 17A and 17B are plots of the data of Tables 12A and 12B, with FIG. 17A plotting all of the data of these tables and FIG. 17B plotting Day +3 data for tail flick testing doses of 0, 0.25, 0.5, 0.75, and 1.0 mg/kg.

### VI. DEFINITIONS

As used in this specification and in the claims, the following terms and phrases shall have the following meanings.

By “dosage form” is meant a pharmaceutical composition or device comprising an active pharmaceutical agent, such as oxycodone and/or one or more of its pharmaceutically-acceptable acid addition salts, the composition or device also containing inactive ingredients, i.e., pharmaceutically acceptable excipients such as suspending agents, surfactants, disintegrants, binders, diluents, lubricants, stabilizers, antioxidants, osmotic agents, colorants, plasticizers, coatings and the like, that are used to manufacture and deliver active pharmaceutical agents.

By “active agent”, “drug”, or “compound” is meant an agent, drug, or compound having the characteristics of oxycodone and/or one or more of its pharmaceutically-acceptable acid addition salts. If desired, other analgesics or, more generally, other medicaments, can be included in the dosage forms of the invention.

By “pharmaceutically-acceptable acid addition salts” are meant those salts in which the anion does not contribute significantly to the toxicity or pharmacological activity of the salt, and, as such, they are the pharmacological equivalents of the bases of the oxycodone compound. Examples of pharmaceutically acceptable acids that are useful for the purposes of salt formation include but are not limited to hydrochloric, hydrobromic, hydroiodic, citric, acetic, benzoic, mandelic, phosphoric, nitric, mucic, isethionic, palmitic, and others.

By “sustained release” is meant predetermined substantially continuous release of active agent to an environment over a prolonged period.

The expressions “exit,” “exit orifice,” “delivery orifice” or “drug delivery orifice,” and other similar expressions, as may be used herein include one or more members selected from the group consisting of a passageway; an aperture; an orifice; and a bore. The expressions also include orifices that are formed or formable from a substance or polymer that erodes, dissolves or is leached from the dosage form to thereby form an exit orifice.

A drug “release rate” refers to the quantity of drug released from a dosage form per unit time, e.g., milligrams of drug released per hour (mg/hr). Drug release rates for drug dosage forms are typically measured as an in vitro rate of release, i.e., a quantity of drug released from the dosage form per unit time measured under appropriate conditions and in a suitable fluid. The release rate tests utilized in the examples described herein were performed on dosage forms placed in metal coil sample holders attached to a USP Type VII bath indexer in a constant temperature water bath at 37° C. Aliquots of the release rate solutions were injected into a chromatographic system to quantify the amounts of drug released during the testing intervals.

By “release rate assay” is meant a standardized assay for the determination of the release rate of a compound from a dosage form tested using a USP Type 7 interval release apparatus. It is understood that reagents of equivalent grade may be substituted in the assay in accordance with generally accepted procedures.

For clarity and convenience herein, the convention is utilized of designating the time of drug administration as zero hours (t=0 hours) and times following administration in appropriate time units, e.g., t=30 minutes or t=2 hours, etc. As used herein, unless otherwise specified, a drug release rate obtained at a specified time “following administration” refers to the in vitro drug release rate obtained at the specified time following implementation of an appropriate dissolution test. The time at which a specified percentage of the drug within a dosage form has been released may be referenced as the “Tₓ” value, where “x” is the percent of drug that has been released. For example, a commonly used reference measurement for evaluating drug release from dosage forms is the time at which 70% of drug within the dosage form has been released. This measurement is referred to as the “T₇₀” for the dosage form.

An “immediate-release dosage form” refers to a dosage form that releases drug substantially completely within a short time period following administration, i.e., generally within a few minutes to about 1 hour.

By “sustained release dosage form” is meant a dosage form that releases drug substantially continuously for many hours (the “sustained release time period”). Sustained release dosage forms in accord with the present invention preferably exhibit T₇₀ values of at least about 10 to 20 hours and preferably 15 to 18 hours. The dosage forms preferably continuously release drug for sustained periods of at least about 10 hours, more preferably 12 hours or more and, even more preferably, 16-20 hours or more.

Dosage forms in accord with the present invention preferably exhibit uniform release rates of oxycodone for a prolonged period of time within the sustained release time period.

By “uniform release rate” is meant an average hourly release rate from the core that varies positively or negatively by no more than about 30% and preferably no more than about 25% and most preferably no more than 10% from either the preceding or the subsequent average hourly release rate as determined in a USP Type 7 Interval Release Apparatus where the cumulative release is between about 25% to about 75%.

By “prolonged period of time” is meant a continuous period of time of at least about 4 hours, preferably 6-8 hours or more and, more preferably, 10 hours or more. For example, the exemplary osmotic dosage forms described herein generally begin releasing oxycodone at a uniform release rate within about 2 to about 6 hours following administration and the uniform rate of release, as defined above, continues for a prolonged period of time from about 25% to until at least about 75% and preferably at least about 85% of the drug is released from the dosage form. Release of oxycodone continues thereafter for several more hours although the rate of release is generally slowed somewhat from the uniform release rate.

By the phrase “a dosage form having a substantially zero order in vitro release profile” and similar phrases is meant a dosage form which overall has substantially zero order in vitro release kinetics, i.e., the overall in vitro release rate is substantially constant over a 24 hour period. For example, for a dosage form which has both a controlled-release component and an initial loading dose (initial loading component), a substantially zero order in vitro release profile means that the in vitro release rate resulting from the combined release of drug from the two components is substantially constant over a 24 hour period. At steady state, a dosage form that has a substantially zero order in vitro release profile produces an in vivo plasma profile that is substantially flat as opposed to being biphasic as with the OXYCONTIN product (see below).

By “C” is meant the concentration of drug in the blood plasma of a subject, generally expressed as mass per unit volume, typically nanograms per milliliter. For convenience, this concentration may be referred to herein as “plasma drug concentration” or “plasma concentration” which is intended to be inclusive of drug concentration measured in any appropriate body fluid or tissue. The plasma drug concentration at any time following drug administration is referenced as Cₜᵢₘₑ, as in C₉ₕ or C₂₄ₕ, etc.

By “steady state” is meant the condition in which the profile of drug present in the blood plasma of a subject does not vary significantly over a prolonged period of time. A pattern of drug accumulation following continuous administration of a dosage form at constant dosing intervals eventually achieves a “steady-state” where the plasma concentration peaks and plasma concentration troughs are essentially unchanged for each dosing interval.

Persons of skill in the art appreciate that plasma drug concentrations obtained in individual subjects will vary due to intrapatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form dissolution rates and in vivo plasma drug concentrations.

### VII. DETAILED DESCRIPTION OF THE INVENTION AND ITS PREFERRED EMBODIMENTS

A. Dosage Forms

The present invention can be practiced using a variety of techniques known in the art for producing controlled-release oral dosage forms. Non-limiting examples of such techniques include osmotic systems, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems as described in Remington's Pharmaceutical Sciences, 1990 ed., pp. 1682-1685. Oxycodone dosage forms that operate in accord with any of these or other approaches are encompassed by the present invention to the extent that the drug release characteristics and/or the plasma oxycodone concentration characteristics of the appended claims are achieved by those dosage forms either literally or equivalently.

As illustrated by the examples set forth below, particularly preferred dosage forms for use in the practice of the invention are osmotic dosage forms. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. A significant advantage to osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated in its entirety by reference herein. In particular, the following U.S. Patents, owned by ALZA Corporation and directed to osmotic dosage forms, are each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

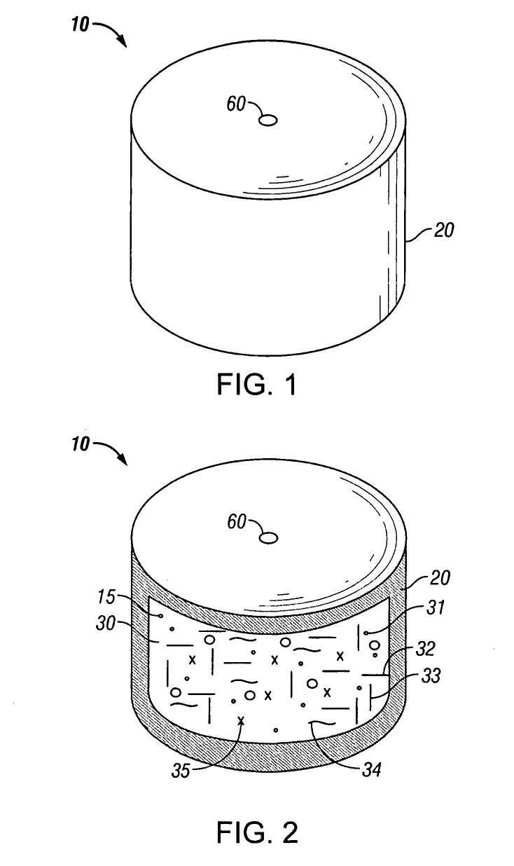

FIG. 1 is a perspective view of one embodiment of a controlled release osmotic dosage form. Dosage form 10 comprises wall 20 that surrounds and encloses an internal compartment (not seen in FIG. 1). The internal compartment contains a composition comprising oxycodone, and/or one or more of its pharmaceutically acceptable acid addition salts. Wall 20 is provided with at least one drug delivery exit 60 for connecting the internal compartment with the exterior environment of use. Accordingly, following oral ingestion of dosage form 10, fluid is imbibed through wall 20 and oxycodone and/or one or more of its pharmaceutically acceptable acid addition salts is released through exit 60.

While the preferred geometrical embodiment in FIG. 1 illustrates a standard biconvex shaped tablet, the geometry may embrace a capsule shaped caplet and other oral dosage forms.

FIG. 2 is a cutaway view of FIG. 1 showing an embodiment of a controlled release osmotic dosage form with internal compartment 15 containing a single component layer referred to herein as drug layer 30, comprising drug 31, i.e., at least oxycodone and/or one or more of its pharmaceutically acceptable acid addition salts, in an admixture with selected excipients adapted to provide an osmotic activity gradient for driving fluid from an external environment through wall 20 and for forming a deliverable drug formulation upon inhibition of fluid. As described in more detail below, the excipients may include a suitable suspending agent, also referred to herein as drug carrier 32, binder 33, lubricant 34 and an osmotically active agent, osmagent 35. In operation, following oral ingestion of dosage form 10, the osmotic activity gradient across wall 20 causes gastric fluid to be imbibed through the wall 20 thereby forming a deliverable drug formulation, i.e., a solution or suspension, within the internal compartment. The deliverable drug formulation is released through exit 60 as fluid continues to enter the internal compartment. As release of the drug formulation occurs, fluid continues to be imbibed thereby driving continued release. In this manner, the drug is released in a sustained and continuous manner over an extended time period.

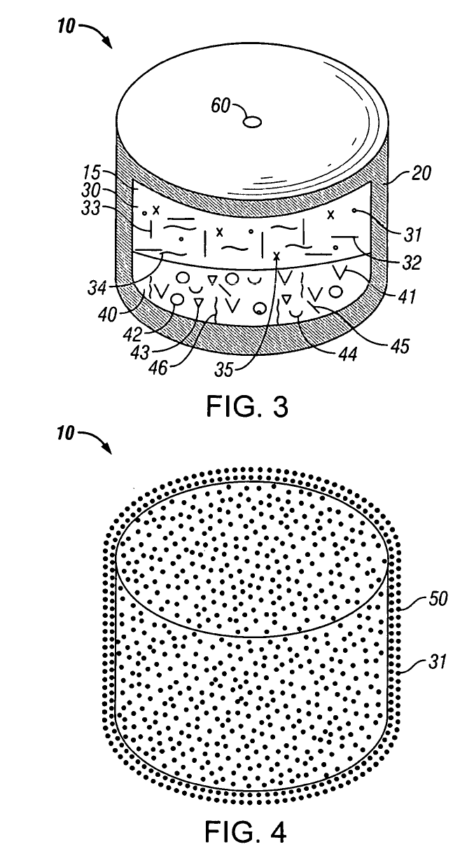

FIG. 3 is a cutaway view of FIG. 1 with an alternate embodiment of internal compartment 15 having a bilayer configuration. In this embodiment, internal compartment 15 contains a bilayered-compressed core having a first component drug layer 30 and a second component push layer 40. Drug layer 30, as described above with reference to FIG. 1, comprises at least oxycodone and/or one or more of its pharmaceutically acceptable acid addition salts in an admixture with selected excipients.

As described in more detail below, second component push layer 40 comprises osmotically active component(s), but does not contain any active agent. The components in push layer 40 typically comprise an osmagent 42 and one or more osmopolymers 41 having relatively large molecular weights which exhibit swelling as fluid is imbibed such that release of these osmopolymers through the drug delivery orifice 60 does not occur. Additional excipients such as binder 43, lubricant 44, antioxidant 45 and colorant 46 may also be included in push layer 40. The second component layer is referred to herein as an expandable or a push layer since, as fluid is imbibed, the osmopolymer(s) swell and push against the deliverable drug formulation of the first component drug layer to thereby facilitate release of the drug formulation from the dosage form.

In operation, following oral ingestion of the dosage form 10 as shown in FIG. 3, the osmotic activity gradient across wall 20 causes gastric fluid to be imbibed through wall 20 thereby forming drug layer 30 into a deliverable formulation and concurrently swelling the osmopolymer(s) in push layer 40. The deliverable drug layer 30 is released through exit 60 as fluid continues to enter internal compartment 15 and push layer 40 continues to swell. As release of drug layer 30 occurs, fluid continues to be imbibed and the push layer continues to swell thereby driving continued release. In this manner, drug is released in a sustained and continuous manner over an extended time period.

Drug layer 30, as described with reference to FIGS. 2 and 3, comprises oxycodone and/or one or more of its pharmaceutically acceptable acid addition salts in an admixture with selected excipients. Push layer 40, as described with reference to FIG. 3, comprises osmotically active component(s) but does not contain any active agent.

Drug layer 30 comprises a composition formed of a pharmaceutically effective amount of oxycodone drug 31, and/or one or more of its pharmaceutically acceptable salts, and a carrier 32. The drug oxycodone is comprised of 4,5-epoxy-14-hydroxy-3-methoxy-17-methylmorphinan-6-one possessing analgesic therapy. Oxycodone is known in the art. The Merck Index, 11^{th} Ed., p. 1100 (1990).

The oxycodone salts are, for example, represented by one or more members selected from the group consisting of the following: oxycodone sulfate, oxycodone hydrochloride, oxycodone trifluoracetate, oxycodone thiosemicarbazone hydrochloride, oxycodone pentafluoropropionate, oxycodone p-nitrophenylhydrozone, oxycodone o-methyloxine, oxycodone thiosemicarbazone, oxycodone semicarbazone, oxycodone phenylhydroazone, oxycodone hydrazone, oxycodone hydrobromide, oxycodone mucate, oxycodone methylbromide, oxycodone oleate, oxycodone n-oxide, oxycodone acetate, oxycodone phosphate dibasic, oxycodone phosphate monobasic, oxycodone inorganic salt, oxycodone organic salt, oxycodone acetate trihydrate, oxycodone bis(heptafluorobutyrate), oxycodone bis(methylcarbamate), oxycodone (bis-pentafluoropropionate), oxycodone bis(pyridine-3-carboxylate), oxycodone bis(trifluoroacetate), oxycodone bitartrate, oxycodone chlorohydrate and oxycodone sulfate pentahydrate.

The dosage form and the therapeutic composition in either manufacture can comprise 1 to 640 mg of oxycodone drug 31 and/or oxycodone drug 31 pharmaceutically acceptable salt. More typically, loading of compound in the dosage forms, whether using osmotic or other controlled-release technology, will provide doses of compound to the subject ranging from 10 mg to 160 mg and more usually 20 mg to 80 mg per day. Generally, if a total drug dose of more than 160 mg per day is required, multiple units of the dosage form may be administered at the same time to provide the required amount of drug. Preferably, the once-a-day dosage forms of the present invention comprise a dose D of oxycodone and/or one or more of its pharmaceutically acceptable acid addition salts that is greater than or equal to about 10 mg and less than or equal to about 80 mg.

For reference, immediate release oxycodone is typically administered at a starting dose of about 10 mg, administered in two or three doses per day. The effective dose range has been determined to be generally 10 mg/day-320 mg/day. Observations of the patient's tolerability to side effects and the need for additional clinical effect over the starting dose often results in the dose being increased in increments of 5 mg/day to 80 mg/day. Concurrently with these observations, plasma concentrations in a subject may be determined by clinical assay to determine a correlation between side effect tolerability, clinical effect, and blood plasma concentrations of the drug. Oxycodone plasma concentrations may range from 0.1 ng/ml to 100 ng/ml (nanograms per milliliter), more typically 4 ng/ml to 40 ng/ml.

For some dosages administered by an osmotic dosage form, it is desirable to modulate the viscosity of the hydrated drug layer in operation by the addition or reduction of salt in the formulation. Traditional systems utilizing salt in a drug formulation dealt with compounds exhibiting a strong common ion effect. This strong common ion effect at high drug loading allowed the addition of salt to modulate the solubility of the compound, allowing more of the salt to be released earlier in the delivery cycle in order to produce a zero order release rate profile. These systems taught incorporation of salt in high drug loading systems with little or no salt in low drug loading systems where a salting out effect was unnecessary.

It has been found that oxycodone and other similar drugs that exhibit a weak common ion effect are not similarly affected by salts to modulate solubility and affect the release rate through a salting out effect. Specifically, it has been found that oxycodone does not benefit from the addition of salt at higher doses, but does benefit from the addition of salt in the low doses. It has also been found that this addition of salt to the lower doses can modulate the viscosity of the hydrated drug layer to maintain a proper delivery for the desired release rate profile.

The amount of salt incorporated into the drug layer of the system is from about 25% if using a high molecular weight polymer and low doses of drug to zero percent if using low molecular weight polymer and higher doses of drug. Representatives of a salt to be incorporated into the drug composition include sodium chloride, potassium chloride and the like. Most preferable is sodium chloride. Preferably, the drug layer viscosity in operation is maintained between about 50 cps and about 100 cps. In this way, products containing lower drug concentrations (5-15%) and higher drug concentrations (15-40%) can essentially be produced such that they have equivalent release functionality.

The drug layer viscosity can be attained by using any of many hydrophilic polymers. Examples include water-soluble cellulose polymers such as NaCMC, HPMC, etc. or polyethylene oxide polymers such as Polyox® or water soluble sugars, such as maltodextrin, sucrose, mannitol. Any physical or chemical property of the polymer, which could be modified to achieve the desired viscosity, is also included in this description.

Carrier 32 may comprise a hydrophilic polymer represented by horizontal dashes in FIGS. 2 and 3. The hydrophilic polymer provides a hydrophilic polymer particle in the drug composition that contributes to the controlled delivery of active agent. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). The drug composition can comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among those polymers are the poly(ethylene oxide) of 100,000-300,000 number average molecular weight. Carriers that erode in the gastric environment, i.e., bioerodible carriers, are especially preferred.

Other carriers that may be incorporated into drug layer 30 include carbohydrates that exhibit sufficient osmotic activity to be used alone or with other osmagents. Such carbohydrates comprise monosaccharide, disaccharides and polysaccharides. Representative examples include maltodextrins (i.e., glucose polymers produced by the hydrolysis of corn starch) and the sugars comprising lactose, glucose, raffinose, sucrose, mannitol, sorbitol, and the like. Preferred maltodextrins are those having a dextrose equivalence (DE) of 20 or less, preferably with a DE ranging from about 4 to about 20, and often 9-20. Maltodextrin having a DE of 9-12 has been found most useful.

Carbohydrates described above, preferably the maltodextrins, may be used in the drug layer 30 without the addition of an osmagent, and obtain the desired release of oxycodone and/or one or more of its pharmaceutically acceptable acid addition salts from the dosage form, while providing a therapeutic effect over a prolonged period of time and up to 24 hours with once-a-day dosing.

The preferred molecular weight of the polymer carrier utilized in the drug layer range from 100,000 mw to 300,000 mw and more preferably about 200,000 mw.

Drug layer 30 may further comprise a therapeutically acceptable vinyl polymer binder 33 represented by vertical dashes in FIG. 2 and FIG. 3. The vinyl polymer comprises a 5,000 to 350,000 average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Dosage form 10 and the therapeutic composition can comprise 0.01 to 25 mg of the binder or vinyl polymer that serves as a binder. Representative of other binders include acacia, starch and gelatin.

Dosage form 30 may further comprise lubricant 34 represented by a wavy line in FIGS. 2 and 3. The lubricant is used during manufacture to prevent sticking to die walls or punch faces. Typical lubricants include magnesium stearate, sodium stearate, stearic acid, calcium stearate, magnesium oleate, oleic acid, potassium oleate, caprylic acid, sodium stearyl fumarate, and magnesium palmitate. The amount of lubricant present in the therapeutic composition can be 0.01 to 10 mg.

Drug layer 30 typically will be a dry composition formed by compression of the carrier and the drug as one layer and the push composition as the other layer in contacting relation.

Drug layer 30 is formed as a mixture containing oxycodone and/or one or more of its pharmaceutically acceptable acid addition salts and the carrier that when contacted with biological fluids in the environment of use provides a slurry, solution or suspension of the compound that may be dispensed by the action of the push layer. The drug layer may be formed from particles by comminution that produces the size of the drug and the size of the accompanying polymer used in the fabrication of the drug layer. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Drug layer 30 may further comprise surfactants and disintegrants. Exemplary of the surfactants are those having an HLB value of between about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like. Disintegrants may be selected from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum and the like.

Push layer 40 comprises a displacement composition in contacting layered arrangement with the first component drug layer 30 as illustrated in FIG. 3. Push layer 40 comprises osmopolymer 41 that imbibes an aqueous or biological fluid and swells to push the drug composition through the exit means of the device. A polymer having suitable inhibition properties may be referred to herein as an osmopolymer. The osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, but in a preferred embodiment are at least lightly crosslinked to create a polymer network that is too large and entangled to exit the dosage form. Thus, in a preferred embodiment, the expandable composition is retained within the dosage form during its operative lifetime.

Push layer 40 comprises 20 to 375 mg of osmopolymer 41, represented by “V” in FIG. 3. Osmopolymer 41 in layer 40 possesses a higher molecular weight than osmopolymer 32 in drug layer 20.

Representatives of fluid-imbibing displacement polymers comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

Push layer 40 can comprise 0 to 75 mg, and presently 5 to 75 mg of an osmotically effective compound, osmagent 42, represented by circles in FIG. 3. The osmotically effective compounds are known also as osmagents and as osmotically effective solutes. Osmagent 42 that may be found in the drug layer and the push layer in the dosage form are those which exhibit an osmotic activity gradient across the wall 20. Suitable osmagents comprise a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts and carbohydrates.

Push layer 40 may further comprise a therapeutically acceptable vinyl polymer 43 represented by triangles in FIG. 3. The vinyl polymer comprises a 5,000 to 350,000 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Push layer can contain 0.01 to 25 mg of vinyl polymer.

Push layer 40 may further comprise 0 to 5 mg of a nontoxic colorant or dye 46, identified by vertical wavy lines in FIG. 3. Colorant 35 includes Food and Drug Administration Colorant (FD&C), such as FD&C No. 1 blue dye, FD&C No. 4 red dye, red ferric oxide, yellow ferric oxide, titanium dioxide, carbon black, and indigo.

Push layer 40 may further comprise lubricant 44, identified by half circles in FIG. 3. Typical lubricants comprise a member selected from the group consisting of sodium stearate, potassium stearate, magnesium stearate, stearic acid, calcium stearate, sodium oleate, calcium palmitate, sodium laurate, sodium ricinoleate and potassium linoleate. The concentration of lubricant can be 0.01 to 10 mg.

Push layer 40 may further comprise an antioxidant 45, represented by slanted dashes in FIG. 3 to inhibit the oxidation of ingredients comprising expandable formulation 40. Push layer 40 can comprise up to 5 mg of an antioxidant. Representative antioxidants comprise a member selected from the group consisting of ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

FIG. 4 depicts a preferred embodiment of the present invention comprising an overcoat 50 of drug 31 on the dosage form of FIG. 3. Overcoat 50 can be a therapeutic composition comprising 0.5 to 75 mg of oxycodone 31 and/or one or more of its pharmaceutically acceptable acid addition salts and 0.5 to 275 mg of a pharmaceutically acceptable carrier selected from the group consisting of alkylcellulose, hydroxyalkylcellulose and hydroxypropylalkylcellulose. For example, the overcoat can contain methylcellulose, hydroxyethylcellulose, hydroxybutylcellulose, hydroxypropylcellulose, hydroxypropylmethylcellulose, hydroxypropylethylcellulose and hydroxypropylbutylcellulose. Overcoat 50 provides therapy immediately as overcoat 50 dissolves or undergoes dissolution in the presence of gastrointestinal fluid and concurrently therewith delivers oxycodone drug 31 and/or one or more of its pharmaceutically acceptable acid addition salts into the gastrointestinal tract for immediate oxycodone therapy.

Exemplary solvents suitable for manufacturing the dosage form components comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Wall 20 is formed to be permeable to the passage of an external fluid, such as water and biological fluids, and it is substantially impermeable to the passage of oxycodone and/or one or more of its pharmaceutically acceptable acid addition salts, osmagent, osmopolymer, and the like. As such, it is semipermeable. The selectively semipermeable compositions used for forming the wall are essentially nonerodible and they are substantially insoluble in biological fluids during the life of the dosage form.

Representative polymers for forming wall 20 comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions typically include a member selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Additional semipermeable polymers for forming the outer wall 20 comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodium styrenesulfonate); semipermeable poly(vinylbenzyltrimethylammonium chloride); and semipermeable polymers exhibiting a fluid permeability of 10⁻⁵ to 10⁻² (cc. mil/cm hr.atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio.

Wall 20 may also comprise a flux-regulating agent. The flux regulating agent is a compound added to assist in regulating the fluid permeability or flux through wall 20. The flux-regulating agent can be a flux-enhancing agent or a flux-decreasing agent. The agent can be preselected to increase or decrease the liquid flux. Agents that produce a marked increase in permeability to fluid such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents may include polyhydric alcohols, polyalkylene glycols, polyalkylenediols, polyesters of alkylene glycols, and the like. Typical flux enhancers include polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Presently preferred flux enhancers include the group of difunctional block-copolymer polyoxyalkylene derivatives of propylene glycol known as pluronics (BASF). Representative flux-decreasing agents include phthalates substituted with an alkyl or alkoxy or with both an alkyl and alkoxy group such as diethyl phthalate, dimethoxyethyl phthalate, dimethyl phthalate, and [di(2-ethylhexyl) phthalate], aryl phthalates such as triphenyl phthalate, and butyl benzyl phthalate; polyvinyl acetates, triethyl citrate, eudragit; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials may be included in the semipermeable wall material for imparting flexibility and elongation properties, to make wall 20 less brittle and to render tear strength. Suitable materials include phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

Pan coating may be conveniently used to provide the completed dosage form, except for the exit orifice. In the pan coating system, the wall-forming composition for wall 20 is deposited by successive spraying of the appropriate wall composition onto the compressed single or bilayered core comprising the drug layer for the single layer core or the drug layer and the push layer for the bilayered core, accompanied by tumbling in a rotating pan. A pan coater is used because of its availability at commercial scale. Other techniques can be used for coating the compressed core. Once coated, the wall can be dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed single or bilayer core in a current of air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent for the wall forming material. An Aeromatic® air-suspension coater can be used employing a cosolvent.

Dosage forms in accord with the present invention are manufactured by standard techniques. For example, the dosage form may be manufactured by the wet granulation technique. In the wet granulation technique, the drug and carrier are blended using an organic solvent, such as denatured anhydrous ethanol, as the granulation fluid. The remaining ingredients can be dissolved in a portion of the granulation fluid, such as the solvent described above, and this latter prepared solution is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for 10 minutes. The composition is pressed into a layer, for example, in a Manesty® press or a Korsch LCT press. For a bilayered core, the drug-containing layer is pressed and a similarly prepared wet blend of the push layer composition, if included, is pressed against the drug-containing layer. The intermediate compression typically takes place under a force of about 50-100 newtons. Final stage compression typically takes place at a force of 3500 newtons or greater, often 3500-5000 newtons. The single or bilayer compressed cores are fed to a dry coater press, e.g., Kilian® Dry Coater press, and subsequently coated with the wall materials as described above.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

In another manufacture the drug and other ingredients comprising the drug layer are blended and pressed into a solid layer. The layer possesses dimensions that correspond to the internal dimensions of the area the layer is to occupy in the dosage form, and it also possesses dimensions corresponding to the second push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. The compressed cores then may be coated with the semipermeable wall material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Exit 60 is provided in each dosage form. Exit 60 cooperates with the compressed core for the uniform release of drug from the dosage form. The exit can be provided during the manufacture of the dosage form or during drug delivery by the dosage form in a fluid environment of use.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

The exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form. The dosage form can be constructed with one or more exits in spaced-apart relation or one or more surfaces of the dosage form.

Drilling, including mechanical and laser drilling, through the semipermeable wall can be used to form the exit orifice. Such exits and equipment for forming such exits are disclosed in U.S. Pat. Nos. 3,916,899, by Theeuwes and Higuchi and in U.S. Pat. No. 4,088,864, by Theeuwes, et al., each of which is incorporated in its entirety by reference herein. It is presently preferred to utilize a single exit orifice.

Techniques corresponding to those described above for osmotic systems are used for dosage forms employing other controlled-release technologies. For example, matrix systems are described in various of the patents relating to Purdue Pharma's OXYCONTIN products. See, for example, U.S. Pat. Nos. 4,861,598; 4,970,075; 5,226,331; 5,508,042; 5,549,912; and 5,656,295. Based on the present disclosure, persons skilled in the art will be readily able to adapt such other controlled-release technologies to produce the in vitro and in vivo profiles of the present invention.

B. Single Dose Cₘₐₓ Values

One of the advantages of the preferred embodiments of the invention is the production of single dose plasma profiles that have small Cₘₐₓ values. Cₘₐₓ values that are large are known to be undesirable for a variety of reasons. For example, high oxycodone concentrations are known to be associated with respiratory depression and resulting high CO₂ levels in the blood. See Leino et al., “Time course of changes in breathing pattern in morphine- and oxycodone-induced respiratory depression,” Anaesthesia, 1999, 54:835-840.

Although specific studies have not been done with oxycodone, “liking” studies have been performed using morphine and have shown higher “liking” values for higher plasma morphine concentrations. See Marsch et al., “Effects of Infusion Rate of Intravenously Administered Morphine on Physiological, Psychomotor, and Self-Reported Measures in Humans,” Journal of Pharmacology and Experimental Therapeutics, 2001, 299:1056-1065. Marsch et al. summarized their findings in this regard at page 1063 of their article as follows: “These results suggest that suggestive measures of drug liking may depend on both the rapidity and magnitude of changes in blood levels of the drug. . . . ” Thus, in and of itself, reducing single dose Cₘₐₓ values represents an important contribution to the art.

As discussed above, the present invention provides substantially zero order (SZO) release profiles. The plasma oxycodone concentration profile for an oral controlled-release dosage form with a constant release rate of R can be modeled using the following equation:

$$C ( t ) = \frac{{k}_{a} \times R}{( {k}_{a} - {k}_{e} ) ( {V}_{d} / F )} [ \frac{1}{{k}_{e}} ( 1 - {ⅇ}^{- {k}_{a} t} ) - \frac{1}{{k}_{a}} ( 1 - {ⅇ}^{- {k}_{a} t} ) ] \qquad Eq . 1$$

where kₐ is an absorption rate constant, kₑ is an elimination rate constant, and V_{d}/F is the mean apparent volume of distribution. kₑ can be derived as the ratio of CL/F to V_{d}/F, where CL/F is the mean apparent clearance.

The plasma oxycodone concentration after a single administration of oxycodone oral solution, 20 mg, has been previously modeled by Mandema, J. W., R. F. Kaiko, B. Oshlack, R. F. Reder and D. R. Stanski (1996). “Characterization and validation of a pharmacokinetic model for controlled release oxycodone,” British journal of clinical pharmacology 42(747-756). The parameters used in this article are set forth in Table 1. Also included in Table 1 are corresponding parameter values derived from the pharmacokinetic data of Examples 5 and 6 below.

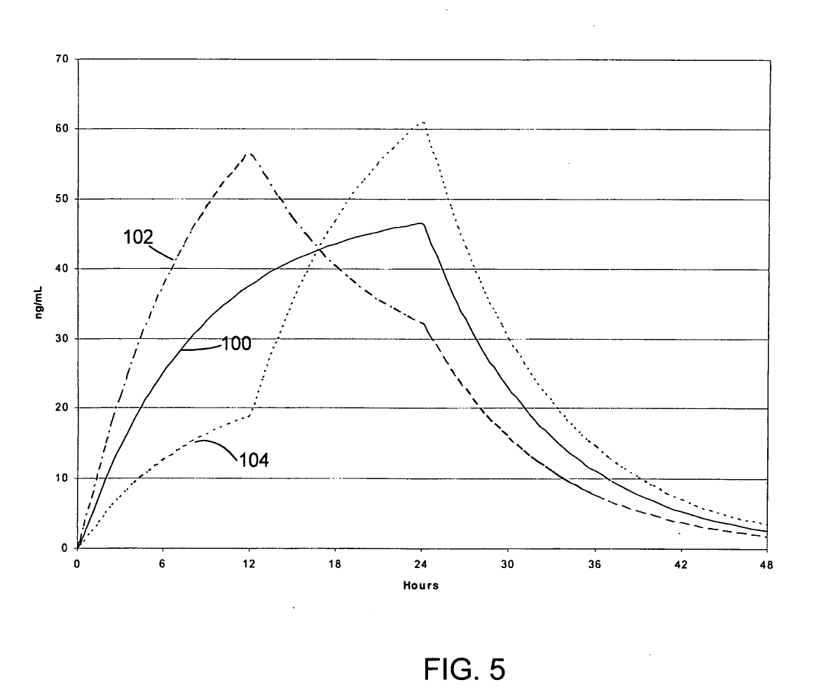

Using the data of Example 6 and Equation 1 above, a single dose profile was calculated for a substantially zero order release rate. The results are shown in FIG. 5 by the curve 100. In addition, two other release profiles were modeled, one having a fast-followed-by-slow release rate and the other having a slow-followed-by-fast release rate. The specific release rates used are set forth in Table 2. Each of these release profiles, as well as the constant release profile used to produce curve 100, released the same amount of drug in 24 hours, i.e., 80 milligrams.

The results of the simulations for the fast-followed-by-slow and slow-followed-by-fast release rates are shown in FIG. 5 by curves 102 and 104, respectively. As can be clearly seen in this figure, each of these curves have higher Cₘₐₓ values than curve 100. The Cₘₐₓ values for curves 102 and 104 are set forth in Table 2. For comparison, the Cₘₐₓ value for curve 100 is 46.5, i.e., 18% lower than the curve 102 value and 24% lower than the curve 104 value.

Although not formally proved, it is believed that the results shown in FIG. 5 will be true of all other profiles, i.e., all profiles which administer the same amount of drug over 24 hours but do not have a constant release rate will have a Cₘₐₓ value larger than that achieved with a constant release rate.

In accordance with the first and seventh aspects of the invention discussed above, Cₘₐₓ for a single dose is specified to be:
3.5×10⁻⁴ liter⁻¹≦Cₘₐₓ/D≦6.8×10⁻⁴ liter⁻¹ Eq. 2
where D is the dose.

The specified upper and lower limits on the Cₘₐₓ-to-dose ratio (Cₘₐₓ/D) in Equation (2) are based on the mean Cₘₐₓ value reported in Table 8 for SZO-24 oxycodone, plus and minus the reported standard deviation for Cₘₐₓ. (Similarly, the upper and lower limits on the AUC₀₋₄₈-to-dose ratio (AUC₀₋₄₈/D) of these aspects of the invention, as well as of the second, third, eighth, and ninth aspects, are based on the mean AUC₀₋₄₈ value for SZO-24 oxycodone reported in Table 8, plus and minus its reported standard deviation.)

Because the data of Table 8 is for a dosage form which had a substantially zero order release rate, based on the modeling of FIG. 5, it is believed that the range for the Cₘₐₓ/D ratio specified in Equation (2) represents the lowest possible range of Cₘₐₓ/D ratios achievable by any oral oxycodone formulation. The provision of dosage forms having such low Cₘₐₓ/D ratios is one of the important contributions to the art of the present invention.

C. Profiles

As discussed above, the present invention provides in vitro dissolution/release profiles and in vivo single dose and steady state plasma profiles for orally-administered oxycodone and/or one or more of its pharmaceutically-acceptable acid addition salts.

Based on how drugs are absorbed and eliminated by the body, the shape of a dosage form's steady state plasma profile is linked to the shape of its single dose plasma profile. In particular, for oxycodone, if one lowers the single dose Cₘₐₓ value while keeping the single dose AUC value substantially the same, the result will be a flatter steady state plasma profile. In terms of the language of the above quoted passage from Benziger et al. 1997, this means that lowering Cₘₐₓ while maintaining AUC will result in “comparatively constant blood levels” of oxycodone. Based on Purdue Pharma's teachings, such blood levels should be avoided because they run the risk of tolerance development.

The AUC₀₋₄₈/D ratio specified in the first, seventh, and other aspects of the invention (i.e., the specification that 7.6×10⁻³ hour/liter≦AUC₀₋₄₈/D≦16.7×10⁻³ hour/liter) is characteristic of how the body absorbs and eliminates oxycodone. Thus, because OXYCONTIN administers its incorporated dose during such time as the dosage form is in the body, its AUC₀₋₄₈/D ratio is within the range for AUC₀₋₄₈/D ratios specified in the first and seventh aspects of the invention. Specifically, as shown in Table 8, OXYCONTIN has a mean AUC₀₋₄₈/D ratio of 12.6×10⁻³ hour/liter ((1007.3 hr-ng/ml)/80 mg=12.6×10⁻³ hour/liter), which is within the specified range of 7.6×10⁻³ to 16.7×10⁻³ hour/liter.

While the specified AUC₀₋₄₈/D values bracket the OXYCONTIN value, the specified upper limit on the single dose Cₘₐₓ/D value, i.e., 6.8×10⁴ liter⁻¹ is significantly below that of OXYCONTIN. Specifically, in connection with the pharmacokinetic study of Example 6, Cₘₐₓ for a single dose of OXYCONTIN 40 mg was found to be 41.8 ng/mL. When divided by 40 mg, the result is 10.5×10⁻⁴/liter, which is well above the specified upper limit of 6.8×10⁻⁴/liter of the first and seventh aspects of the invention.

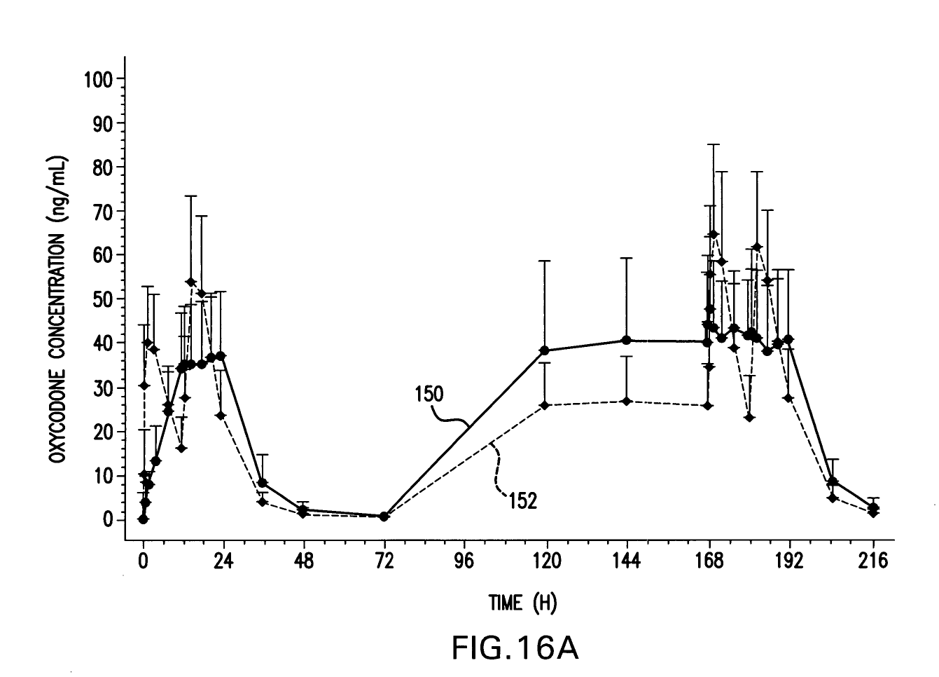

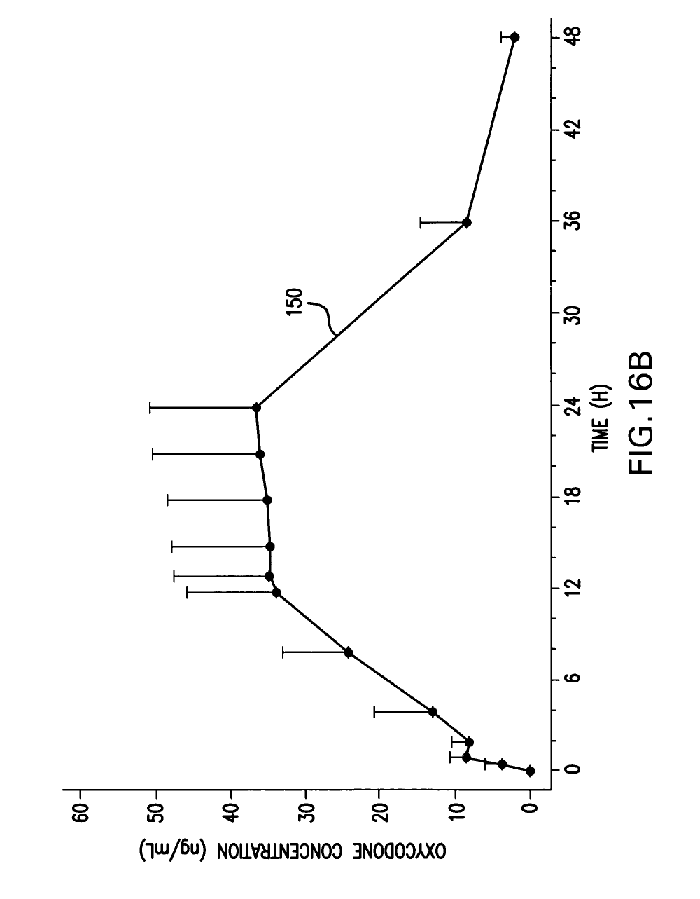

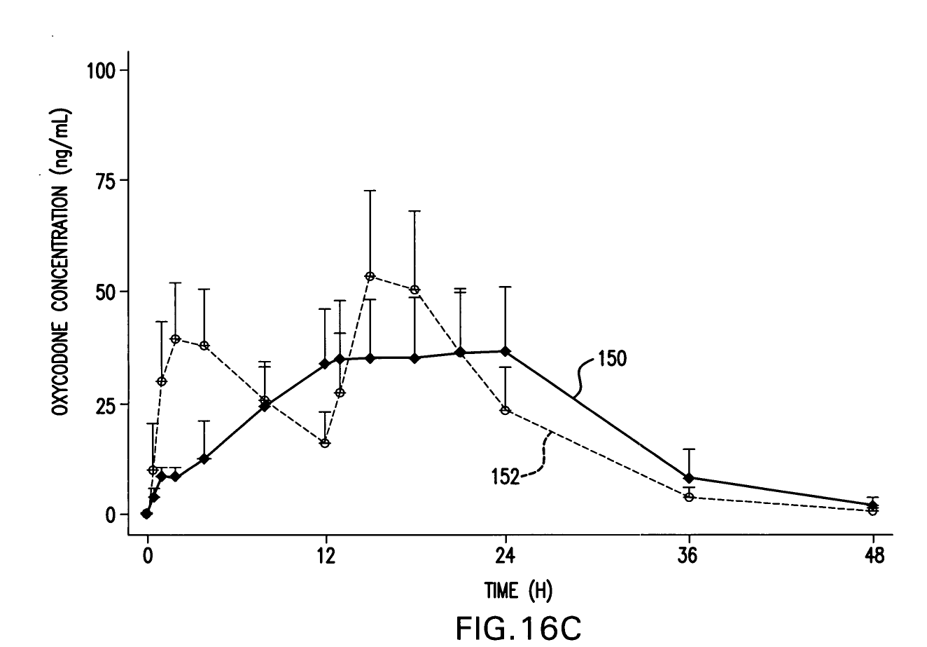

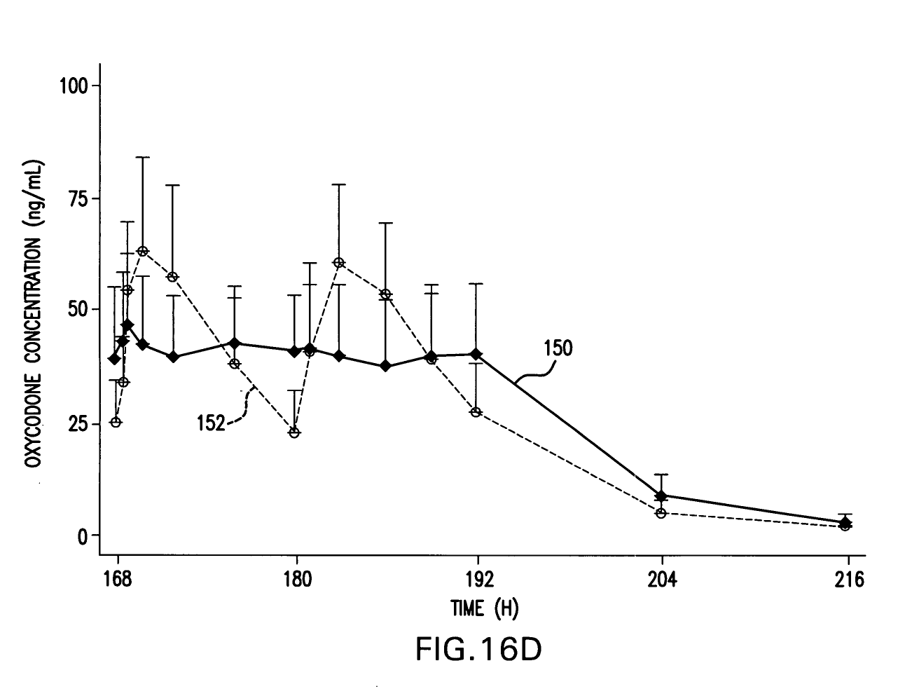

Thus, the first and seventh aspects of the invention specify a single dose AUC value which brackets OXYCONTIN, but a lower Cₘₐₓ. Based on the linkage between single dose and steady state profiles discussed above, this means that a steady state profile is being specified that is generally flatter than that produced by OXYCONTIN. FIG. 16D confirms that this is precisely what is observed. As can be seen in this figure, the SZO-24 steady state profile (curve 150) is almost completely flat while the OXYCONTIN profile (curve 152) clearly oscillates.

Based on the foregoing, it is evident that the single dose profiles specified in the first and seventh aspects of the invention call for a dosage form which is exactly opposite to what Purdue Pharma has taught, namely, that one should not use a dosage form which produces a flat steady state profile because of the risk of tolerance. As discussed fully below (see Example 8), experimentally it has been found that notwithstanding Purdue Pharma's teachings, oxycodone tolerance levels associated with biphasic profiles (i.e., OXYCONTIN type profiles) and flat profiles (i.e., SZO-24 type profiles) are, in fact, not statistically different. This is plainly contrary to what would have been expected based on Purdue Pharma's warnings regarding “comparatively constant blood levels” of oxycodone.

With the foregoing as background, we now turn to a specific discussion of the preferred in vivo steady state, in vivo single dose, and in vitro release profiles of the invention.

### 1. In Vivo Steady State Plasma Profiles

In accordance with certain aspects of the invention, it has been found that effective pain management can be achieved with steady state plasma profiles that are sufficiently flat. As used herein, a steady state plasma profile is sufficiently flat to achieve the pain management benefits of the invention if the ratio of the AUC (area under the curve) for each quartile for the profile to the AUC for the full profile, i.e., the full dosing period of 24-hours, is greater than 0.18 (such a profile is hereinafter referred to as a “>18%/quartile steady state profile”).

As is conventional, the first quartile begins at 0 hours (i.e., the time of administration of the dosage form) and ends at 6 hours, the second quartile begins at 6 hours and ends at 12 hours, the third quartile begins at 12 hours and ends at 18 hours, and the fourth quartile begins at 18 hours and ends at 24 hours. As is also conventional, the plasma profiles are mean profiles obtained from a study population and the AUC values for the quartiles and for the entire profile are obtained using the trapezoidal method. More particularly, the AUC ratios are determined for each individual and then those values are averaged. Samples are taken from subjects in accordance with a sampling scheme selected to reflect the time course of the plasma profile, e.g., there may be more sampling points where the profile is changing rapidly in time.

Preferably, the ratio of the AUC for each quartile of the profile to the AUC for the full profile is greater than or equal to about 0.20. Even more preferably, the difference in ratios between any two adjacent quartiles is less than about 0.03 and/or the difference in ratios between any two quartiles is less than about 0.05. Most preferably, both these criteria are satisfied, i.e., the difference in ratios between any two adjacent quartiles is less than about 0.03 and the difference in ratios between any two quartiles is less than about 0.05.

As the data present below demonstrates, it has been found that >18%/quartile steady state profiles assure efficacy within each quartile, thus reducing the probability of breakthrough pain which has been a long standing problem in pain management using controlled-release dosage forms.

### 2. In Vivo Single Dose Plasma Profiles

In accordance with other aspects of the invention, it has been further found that such desirable >18%/quartile steady state profiles are related to single dose plasma profiles having certain preferred characteristics. One such preferred characteristic of the single dose plasma profile is a mean profile shape which increases substantially monotonically over a period of 24 hours or more.

In certain embodiments, such substantially monotonically increasing mean profile comprises a first rising phase and a second phase, where the slope of the first phase is greater than the magnitude of the slope of the second phase, where the slope of a phase is defined as the slope of a best fit straight line to the portion of the mean profile making up the phase. For example, the slope of the first phase can be at least approximately 10 times the magnitude of the slope of the second phase. In other embodiments, the first rising phase can include a first rising subphase followed by a second rising subphase, where the slope of the first rising subphase is greater than the slope of the second rising subphase, where slopes are defined in the same manner as for the first and second phases.

Generally, the transition from the first phase to the second phase occurs at about 14 hours, e.g., between about 12 hours and about 16 hours, while the transition from the first subphase to the second subphase occurs at about 2 hours, e.g., between about 1 hour and about 3 hours.

The single dose plasma profiles also preferably have their maximum concentration values (Cₘₐₓ) at a time (Tₘₐₓ) which is greater than about 17 hours, more preferably greater than about 18 hours, and most preferably greater than about 19 hours.

The single dose plasma profiles also preferably have a 12-24 hour AUC which is greater than their 0-12 hour AUC. In particular, the ratio of the 12-24 hour AUC to the 0-12 hour AUC is preferably greater than about 1.5, more preferably greater than about 1.7, and most preferably about 2.0.

To reduce the probability of the dosage form having “liking” problems, the single dose plasma profile preferably has a Cₘₐₓ/(Tₘₐₓ×dose) ratio which is less than about 3×10⁻⁴ hour⁻¹ liter⁻¹, more preferably less than about 4×10⁻⁵ hour-⁻¹ liter⁻¹, and most preferably less than about 3×10⁻⁵ hour⁻¹ liter⁻¹. In this way, the user of the dosage form does not achieve an early, strong bolus of oxycodone and thus is less likely to experience the euphoria and other effects which can lead to a liking response. For comparison, the commercial OXYCONTIN product, which is known to suffer from a liking, indeed, an abuse, problem, has a Cₘₐₓ/(Tₘₐₓ×dose) ratio of about 4×10⁻⁴ hour⁻¹ liter⁻¹ for its 40 mg dosage strength.

As with the steady state profiles, the single dose profiles are mean profiles obtained from a study population and the sampling scheme is selected to reflect the time course of the single dose plasma profile. As discussed above, the slopes are determined from the mean profiles. However, Tₘₐₓ, Cₘₐₓ, and Cₘₐₓ/(Tₘₐₓ×dose) ratios are obtained for individual subjects and then averaged.

### 3. In Vitro Release Profiles

In accordance with other aspects of the invention, it has been further found that the desired >18%/quartile steady state profiles are related to the in vitro dissolution/release profile of the dosage form. In particular, the in vitro dissolution/release profile preferably comprises an initial loading dose component and a controlled release component.

Preferably, the ratio of the amount of oxycodone in the initial loading dose to the total amount of oxycodone in the dosage form is less than 0.25, more preferably less than 0.10, and most preferably less than or equal to 0.05. The 0.25 upper limit on initial loading dose ensures that the dosage form does not generate plasma concentrations above those produced by an immediate release dosage form administered at an equivalent daily dose, and thus the probability of the dosage form having “liking” problems or other adverse side effects will be no worse than for an immediate release product. The 0.10 and 0.05 levels should make such “liking” and other problems even less.

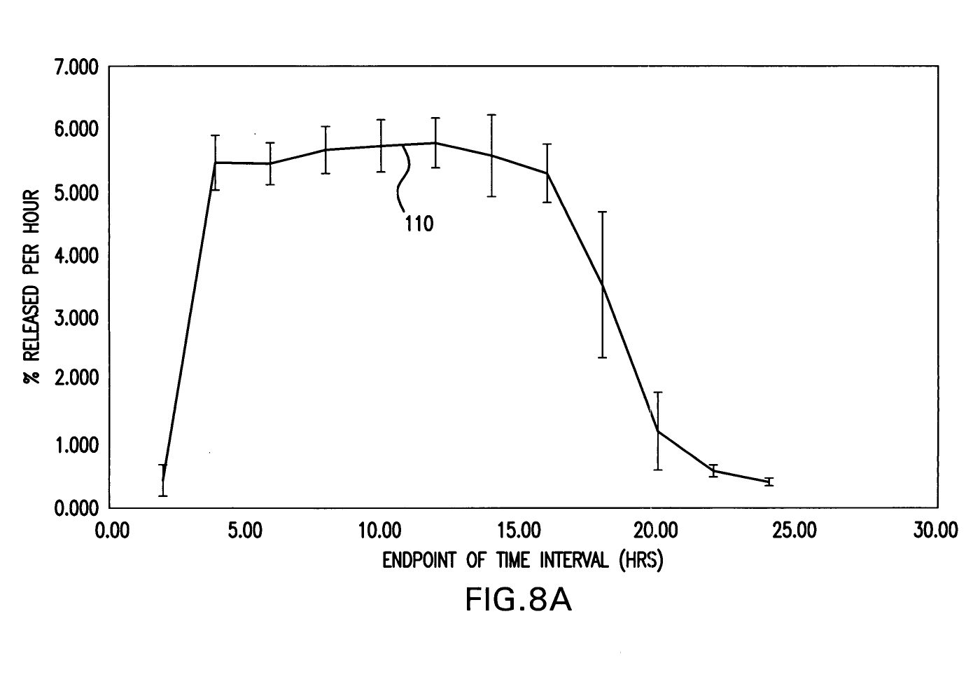

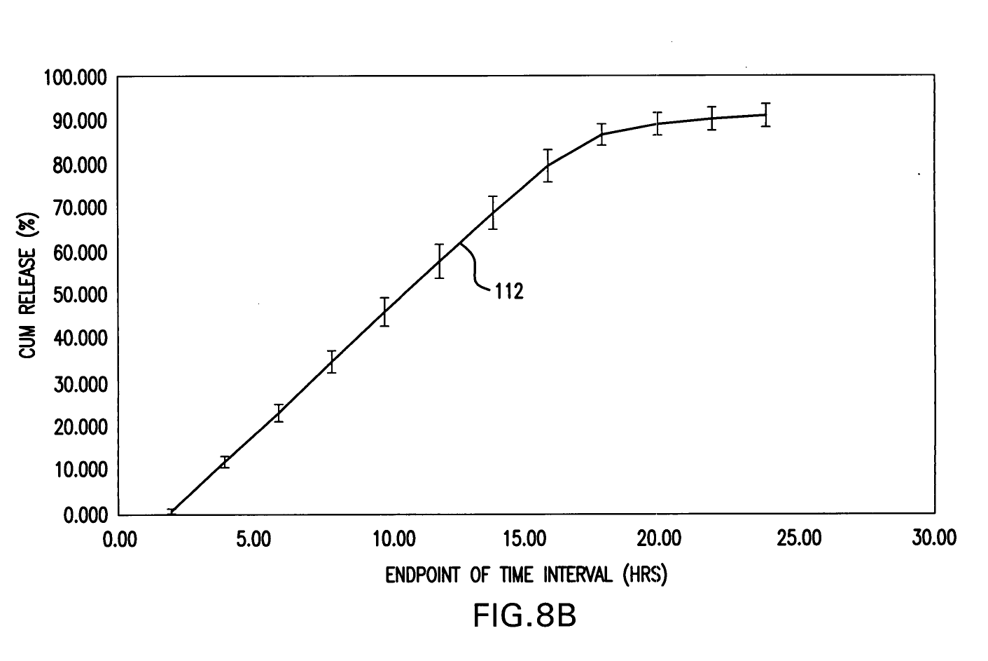

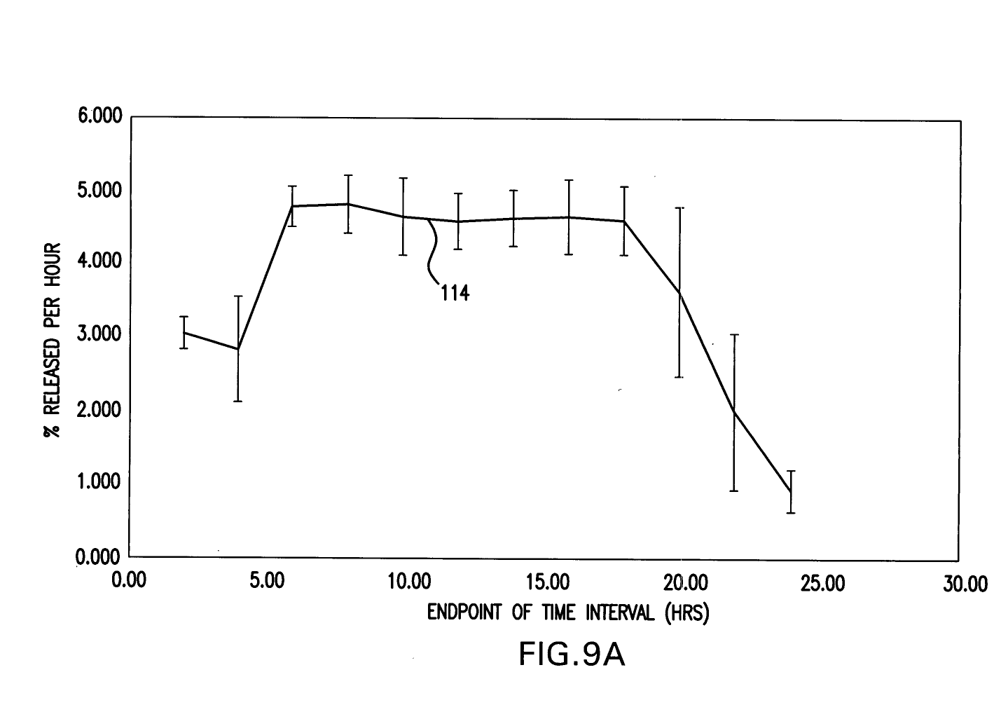

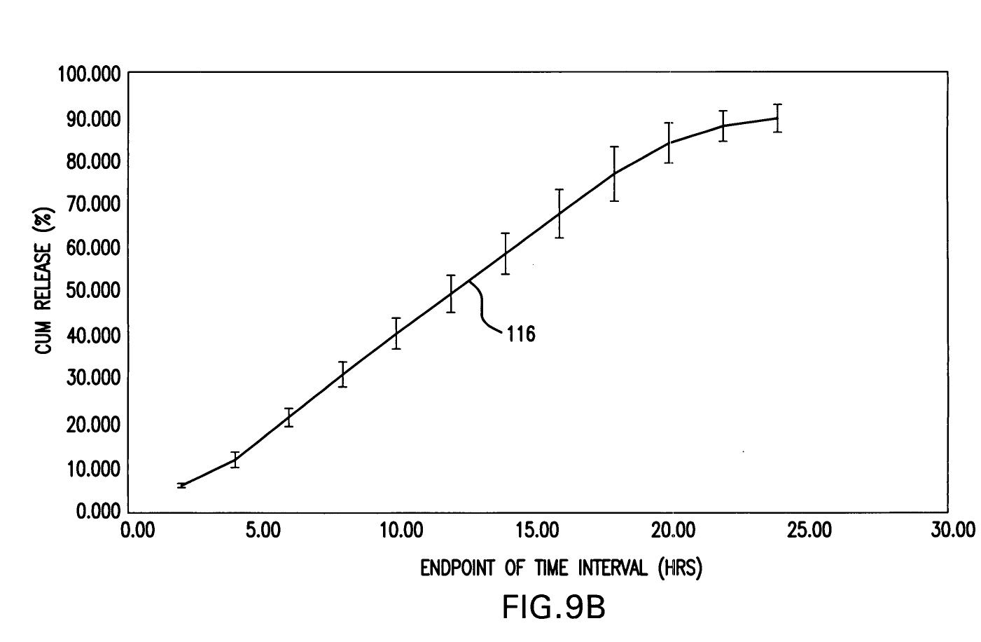

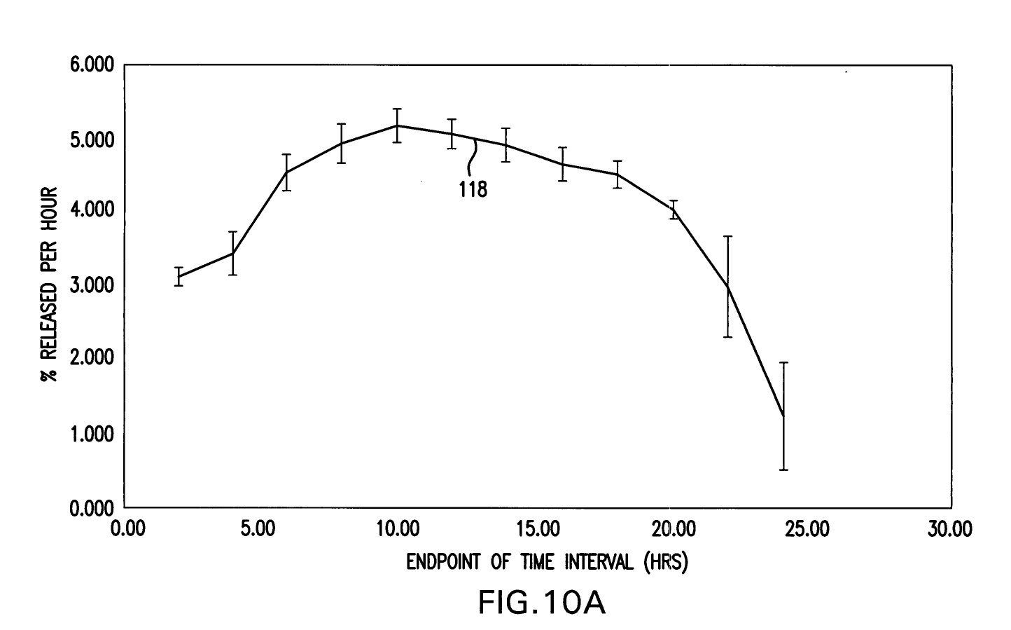

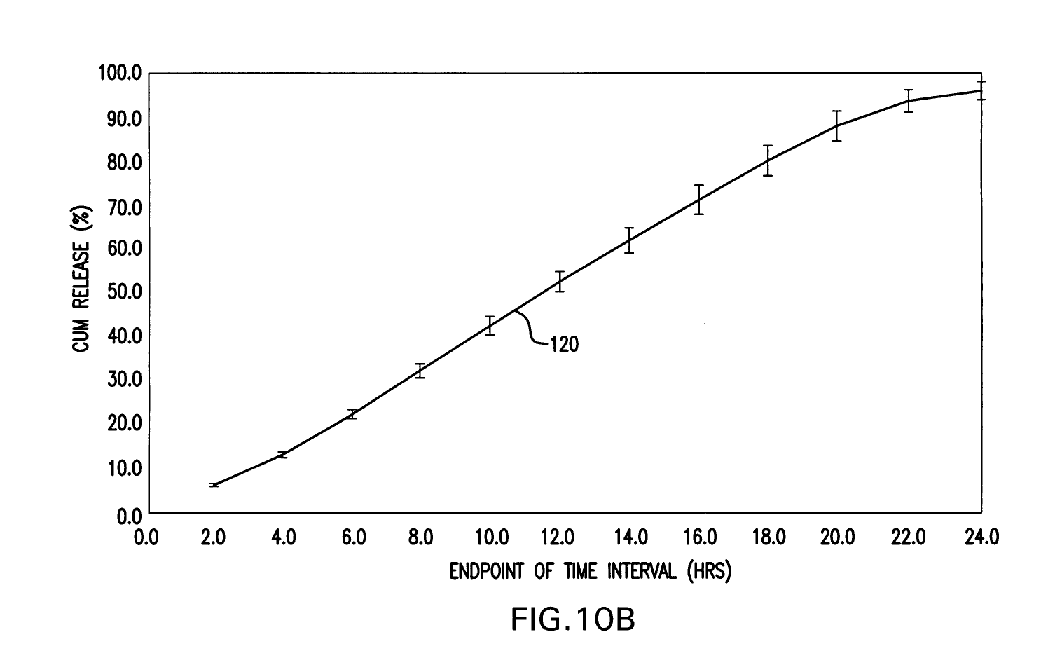

The controlled release component preferably has a substantially constant in vitro dissolution/release rate so that when combined with the initial loading dose, the overall dosage form has substantially zero order in vitro release kinetics, i.e., the overall in vitro release rate is substantially constant over a 24 hour period. FIGS. 9 and 10 are non-limiting examples of release profiles for dosage forms which employ a controlled-release component and an initial loading dose and exhibit substantially zero order in vitro release kinetics, while FIG. 8 is an example of a release profile for a dosage form which achieves those kinetics with only a controlled-release component.

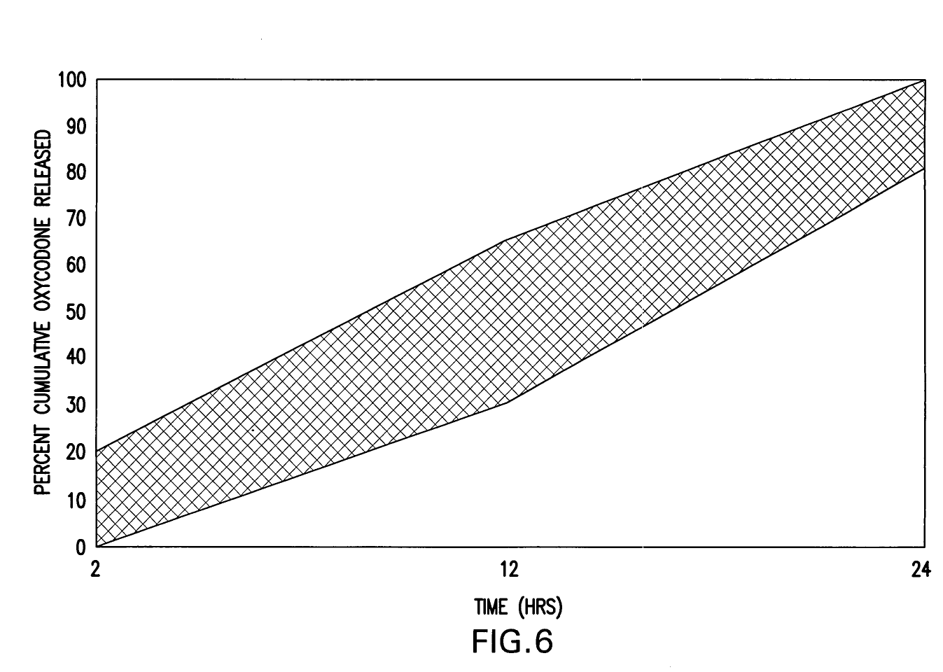

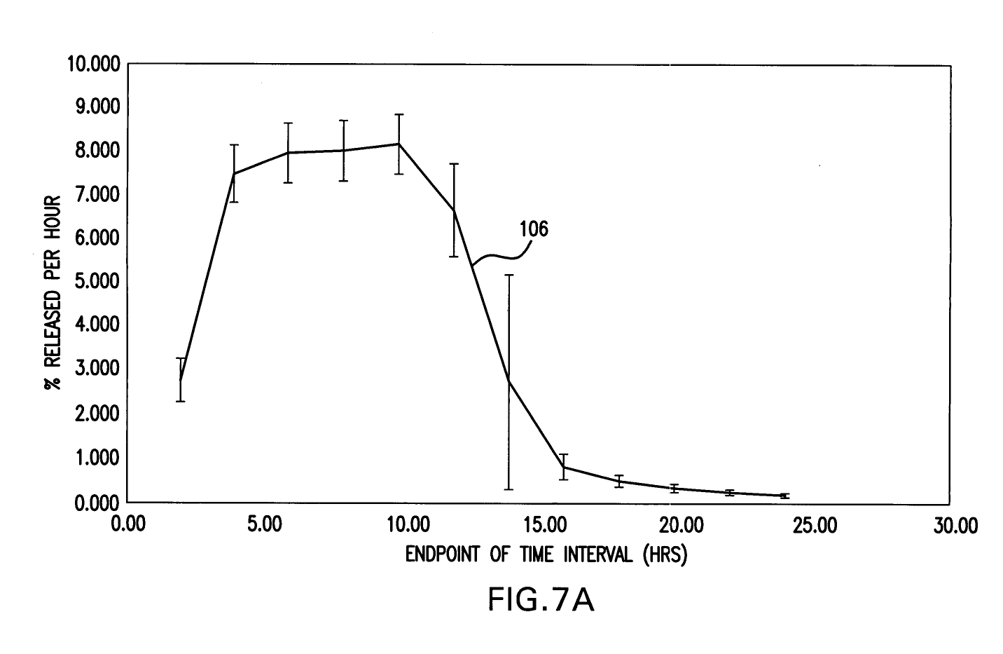

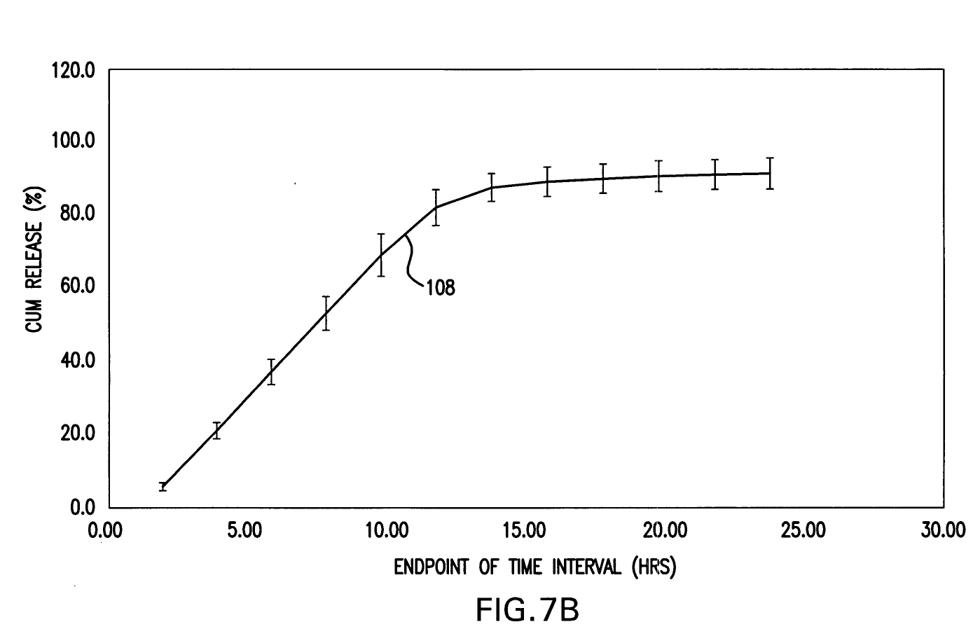

Preferably, the dosage form releases 70% of the dosage form's label dose within a period (the T₇₀ period) of between about 15 hours to about 18 hours. More particularly, the dosage form preferably has a delivery dose pattern of from 0% to 20% in 0-2 hours, 30 to 65% (preferably 33 to 63%) in 0 to 12 hours, and 80 to 100% in 0 to 24 hours, as shown schematically in FIG. 6.

As is conventional, mean in vitro dissolution/release profiles are used which are determined by testing a sample set of dosage forms using USP apparatus 1, 2, or 7, or comparable apparatus which may be substituted in the future. T₇₀ values, however, are an average of the T₇₀ values for the individual dosage forms tested, and similarly the delivery dose pattern for a dosage form is determined by averaging the results for the individual dosage forms tested.

### D. EXAMPLES

The following non-limiting examples illustrate various of the features of the invention.

### Example 1

### Oxycodone Hydrochloride 17 mg Osmotic Push Pull Systems (Fast and Slow)

A dosage form adapted, designed and shaped as an osmotic drug delivery device was manufactured as follows: Two granulations were made by the following procedure: 1479 g of oxycodone hydrochloride, USP and 7351 g of polyethylene oxide N80 with average molecular weight of 200,000 were added to a fluid bed granulator bowl. Next a binder solution was prepared by dissolving 500 g of polyvinylpyrrolidone identified as K29-32 in 4500 g of water. The dry materials were fluid bed granulated by spraying with 1800 g of binder solution. Next, the wet granulation was dried in the granulator to an acceptable moisture content. The two granulations were then sized by passing through a 7-mesh screen into the same container. Next, the granulation was transferred to a blender and mixed with 3.53 g of butylated hydroxytoluene as an antioxidant and lubricated with 88 g of magnesium stearate.

Next, a push composition was prepared as follows: first, a binder solution was prepared. 27.3 kg of polyvinylpyrrolidone identified as K29-32 having an average molecular weight of 40,000 was dissolved in 182.7 kg of water. Then, 22.4 kg of sodium chloride and 1.12 kg of ferric oxide were sized using a Quadro Comil with a 21-mesh screen. Then, the screened materials and 82.52 kg of polyethylene oxide (approximately 2,000,000 molecular weight) were added to a fluid bed granulator bowl. The dry materials were fluidized and mixed while 43 kg of binder solution was sprayed from 3 nozzles onto the powder. The granulation was dried in the fluid-bed chamber to an acceptable moisture level. The granulation process was repeated four times and the granulations were blended together during sizing. The coated granules were sized using a Fluid Air mill with a 7-mesh screen. The granulations were transferred to a tote tumbler, mixed with 224 g of butylated hydroxytoluene and lubricated with 1.12 kg stearic acid.

Next, the oxycodone hydrochloride drug composition and the push composition were compressed into bilayer tablets. First, 113 mg of the oxycodone hydrochloride composition was added to the die cavity and pre-compressed; then, 103 mg of the push composition was added and the layers were pressed into a 5/16″ diameter round, standard concave, bilayer arrangement.

The bilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 20 mg of membrane was applied to each tablet to create “fast” systems. The coating process was repeated and approximately 30 mg of membrane was applied to each tablet to create “slow” systems.

Next, one 25 ml (0.64 mm) exit passageway was laser drilled through the semi-permeable wall to connect the drug layer with the exterior of the dosage system. The residual solvent was removed by drying for 48 hours as 45° C. and 45% humidity followed by 4 hours at 45° C. to remove excess moisture.

The dosage forms produced by this manufacture were designed to deliver 17 mg of oxycodone HCl, USP from the core containing 15.8% oxycodone hydrochloride USP, 81.68% polyethylene oxide N80 possessing a 200,000 molecular weight, 2% polyvinylpyrrolidone possessing a 40,000 molecular weight, 0.02% butylated hydroxytoluene, and 0.5% magnesium stearate. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi-permeable wall comprised 99% cellulose acetate of 39.8% acetyl content and 1% polyethylene glycol. The dosage forms comprised one passageway, 25 mils (0.64 mm) on the center of the drug side. The final dosage forms had a mean release rate of 1.35 mg oxycodone hydrochloride, USP per hour (7.95%/hr) and 0.97 mg oxycodone hydrochloride USP per hour (5.70%/hr) for the “fast” and “slow” systems, respectively.

The formulation ofthis example is summarized in Table 3.

### Example 2

### Oxycodone Hydrochloride 20 ml Osmotic Push Pull System

A dosage form adapted, designed and shaped as an osmotic drug delivery device was manufactured as follows: 1933 g of oxycodone hydrochloride, USP, 7803 g of polyethylene oxide N80 with average molecular weight of 200,000, and 200 g of polyvinylpyrrolidone identified as K29-32 having an average molecular weight of 40,000 were added to a fluid bed granulator bowl. Next a binder solution was prepared by dissolving 500 g of the same polyvinylpyrrolidone in 4500 g of water. The dry materials were fluid bed granulated by spraying with 2000 g of binder solution. Next, the wet granulation was dried in the granulator to an acceptable moisture content, and sized by passing through a 7-mesh screen. Next, the granulation was transferred to a blender and mixed with 2 g of butylated hydroxytoluene as an antioxidant and lubricated with 25 g of magnesium stearate.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of polyethylene oxide (approximately 2,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the oxycodone hydrochloride drug composition and the push composition were compressed into bilayer tablets. First, 113 mg of the oxycodone hydrochloride composition was added to the die cavity and pre-compressed; then, 103 mg of the push composition was added and the layers were pressed into a 5/16″ diameter round, standard concave, bilayer arrangement.

The bilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 37 mg of membrane was applied to each tablet.

Next, one 40 mil (1 mm) exit passageway was laser drilled through the semi-permeable wall to connect the drug layer with the exterior of the dosage system. The residual solvent was removed by drying for 48 hours as 45° C. and 45% humidity. After drilling, the osmotic systems were dried for 4 hours at 45° C. to remove excess moisture.

Next, the drilled and dried systems were coated with an immediate release drug overcoat. The drug overcoat was an 8% solids aqueous solution containing 157.5 g of oxycodone HCl, USP and 850 g of hydroxypropyl methylcellulose possessing an average molecular weight of 11,200. The drug overcoat solution was sprayed onto the dried coated cores until an average wet coated weight of approximately 8 mg per system was achieved.

Next, the drug-overcoated systems were color overcoated. The color overcoat was a 12% solids suspension of Opadry in water. The color overcoat suspension was sprayed onto the drug overcoated systems until an average wet coated weight of approximately 8 mg per system was achieved.

Next, the color-overcoated systems were clear coated. The clear coat was a 5% solids solution of Opadry in water. The clear coat solution was sprayed onto the color coated cores until an average wet coated weight of approximately 3 mg per system was achieved. Next, clear-coated systems were coated with approximately 1 g of Camuaba wax by dispersing the wax over the systems as they tumbled in the pan coater.

The dosage form produced by this manufacture was designed to deliver 1 mg of oxycodone hydrochloride USP as an immediate release from an overcoat comprised of 15% oxycodone HCl, USP and 85% hydroxypropyl methylcellulose followed by the controlled delivery of 19 mg of oxycodone HCl, USP from the core containing 17.7% oxycodone hydrochloride USP, 78.03% polyethylene oxide possessing a 200,000 molecular weight, 4% polyvinylpyrrolidone possessing a 40,000 molecular weight, 0.02% butylated hydroxytoluene, and 0.25% magnesium stearate. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall comprised 99% cellulose acetate of 39.8% acetyl content and 1% polyethylene glycol. The dosage form comprised one passageway, 40 mils (1 mm) on the center of the drug side. The final dosage form contained a color overcoat, a clear overcoat and a wax coat and had a mean release rate of 0.93 mg oxycodone hydrochloride, USP per hour (4.66%/hr).

The formulation of this example is summarized in Table 4 and is referred to hereinafter as the “Example 2 SZO-24 dosage form.”

### Example 3

### Oxycodone Hydrochloride 80 mg Osmotic Push Pull System

A dosage form adapted, designed and shaped as an osmotic drug delivery device was manufactured as follows: 34.36 kg of oxycodone hydrochloride, USP, 63.7 kg of polyethylene oxide N150 with average molecular weight of 200,000, and 0.02 kg of ferric oxide red, were added to a fluid bed granulator bowl. Next, a binder solution was prepared by dissolving 5.40 kg of polyvinylpyrrolidone identified as K29-32 having an average molecular weight of 40,000 in 49.6 kg of water. The dry materials were fluid bed granulated by spraying with 33.3 kg of binder solution. Next, the wet granulation was dried in the granulator to an acceptable moisture content, and sized by passing through a 7-mesh screen. The granulation was then transferred to a blender and mixed with 0.02 kg of butylated hydroxytoluene as an antioxidant and lubricated with 0.25 kg of magnesium stearate.

Next, a push composition was prepared as follows: First, a binder solution was prepared by dissolving 7.8 kg of polyvinylpyrrolidone identified as K29-32 having an average molecular weight of 40,000 in 52.2 kg of water. Then, 24 kg of sodium chloride and 1.2 kg of ferric oxide were sized using a Quadro Comil with a 21-mesh screen. The sized materials and 88.5 kg of polyethylene oxide (approximately 2,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 24 g of butylated hydroxytoluene and lubricated with 300 g magnesium stearate.

Next, the oxycodone hydrochloride drug composition and the push composition were compressed into bilayer tablets. First, 250 mg of the oxycodone hydrochloride composition was added to the die cavity and pre-compressed, then 192 mg of the push composition was added and the layers were pressed into a 13/32″ (1.03 cm) diameter round, standard concave, bilayer arrangement.

The bilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) solvent mixture to make a 5% solids solution. The wall-forming composition was sprayed onto and around the bilayered arrangements in a pan coater until approximately 44 mg of membrane was applied to each tablet.

Next, two 40 mil (1 mm) exit passageways were laser drilled through the semi-permeable wall to connect the drug layer with the exterior of the dosage system. The residual solvent was removed by drying for 72 hours as 45° C. and 45% humidity followed by 4 hours at 45° C. to remove excess moisture.

Next, the drilled and dried systems were coated with an immediate release drug overcoat. The drug overcoat was a 12% solids aqueous solution containing 1.33 kg of oxycodone HCl, USP and 7.14 kg of Opadry™ Clear. The drug overcoat solution was sprayed onto the coated systems until an average wet coated weight of approximately 27 mg per system was achieved.

Next, the drug-overcoated systems were color overcoated. The color overcoat was a 12% solids suspension of Opadry in water. The color overcoat suspension was sprayed onto the drug overcoated systems until an average wet coated weight of approximately 8 mg per system was achieved.

Next, the color-overcoated systems were coated with approximately 100 ppm of Carnuaba wax by dispersing the wax over the systems as they tumbled in the pan coater.

The dosage form produced by this manufacture was designed to deliver 4 mg of oxycodone hydrochloride USP as an immediate release from an overcoat comprised of 15% oxycodone HCl, USP and 85% Opadry™ Clear followed by the controlled delivery of 76 mg of oxycodone HCl, USP from the core containing 32% oxycodone hydrochloride USP, 63.73% polyethylene oxide N150 possessing a 200,000 molecular weight, 4% polyvinylpyrrolidone possessing a 40,000 molecular weight, 0.02% butylated hydroxytoluene, and 0.25% magnesium stearate. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall comprised of 99% cellulose acetate of 39.8% acetyl content and 1% polyethylene glycol. The dosage form comprised two passageways, 40 mils (1 mm) equidistant on the center of the drug side. The final dosage form contained a color overcoat and a wax coat and had a mean release rate of 3.94 mg oxycodone hydrochloride, USP per hour (4.93%/hr).

The formulation of this example is summarized in Table 5 and is referred to hereinafter as the “Example 3 SZO-24 dosage form.”

### Example 4

### Pharmacokinetics and Pharmacodynamics of Osmotic Oxycodone Hydrochloride (Fast and Slow) and Immediate Release Oxycodone Hydrochloride in Healthy Volunteers

This study investigated the pharmacokinetics and pharmacodynamics of the “fast” and “slow” osmotic oxycodone HCl systems of Example 1 and immediate release (IR) oxycodone HCl in healthy male volunteers. In particular, this single-center, randomized, three-treatment, three-period, single- and multiple-dose, crossover, pharmacokinetic/pharmacodynamic study compared two osmotic oxycodone HCl formulations and IR oxycodone HCl (Oxynorm® capsule, 5 mg supplied by Napp Pharmaceuticals, Cambridge Science Park, Milton Rd., Cambridge, United Kingdom) in healthy male subjects over four days. The pharmacodynamic portion of the study was single blind and utilized a VAS pain score. Eighteen subjects enrolled and 15 completed all study periods. While operating, the fast-release and slow-release osmotic dosage forms released oxycodone in a zero-order fashion with different durations and neither dosage form had an immediate-release oxycodone overcoat.

Subjects each received three treatments according to a randomly assigned sequence:
one 17-mg dose of the fast-release dosage form (delivered over approximately 10 hours);
one 17-mg dose of the slow-release dosage form (delivered over approximately 20 hours);
four 5-mg doses of IR oxycodone HCl (one dose at hours 0, 6, 12, and 18 of the study period).

The fast-release formulation produced a larger reduction in the pain score than either the slow-release formulation or IR oxycodone HCl. The reduction in pain scores with the slow-release formulation were generally comparable to those seen with IR oxycodone HCl.

On average, the fast-release and the slow-release formulations were 105% and 99% bioavailable, respectively, relative to IR oxycodone HCl. The plasma oxycodone concentration profiles for the fast and slow formulations were consistent with their in vitro release rate data.

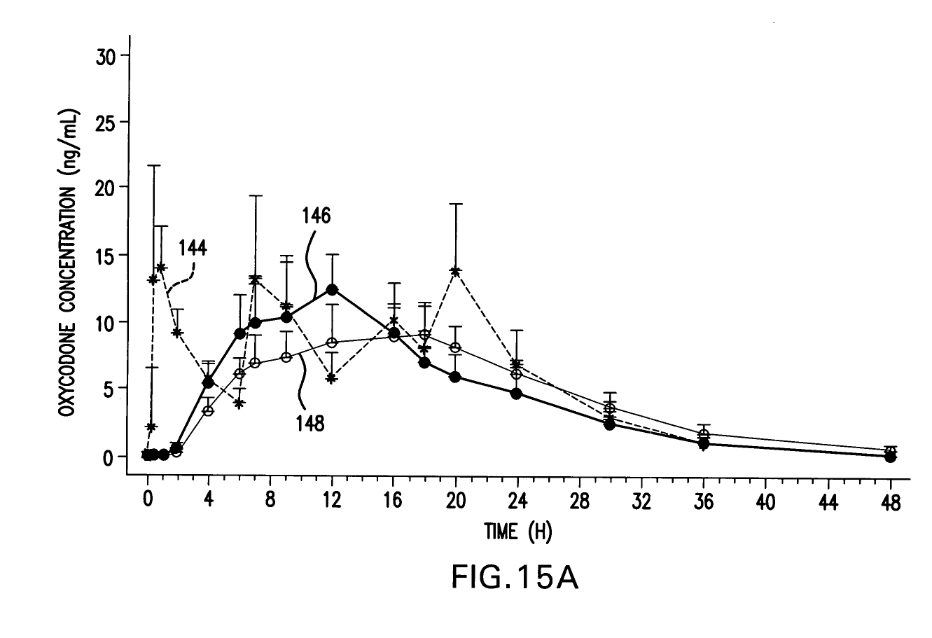

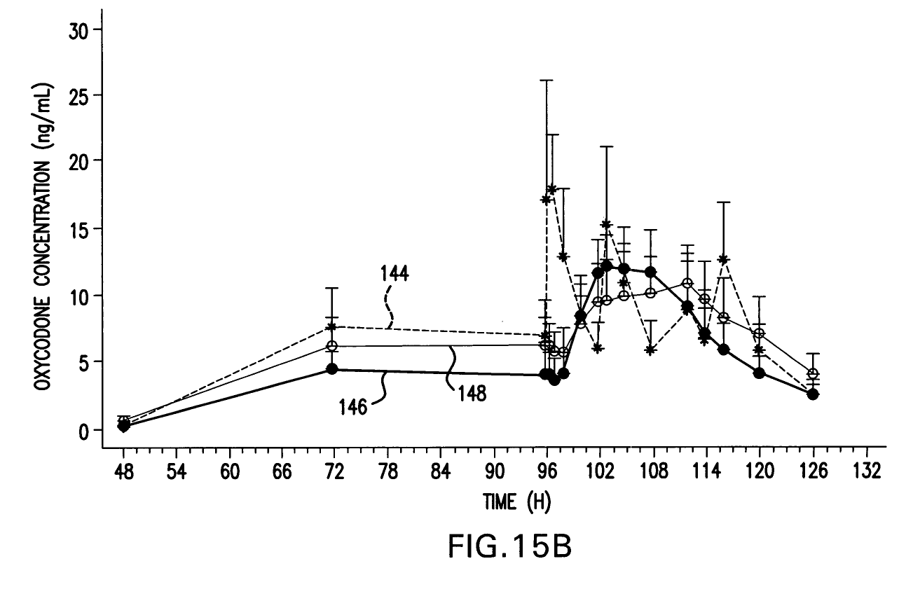

The mean plasma oxycodone concentration profiles after a single day dosing are shown in FIG. 15A. After a single dose administration, the mean Cₘₐₓ/(Tₘₐₓ*Dose) ratio was 7×10⁻⁵ (h*Liter)⁻¹ and 4×10⁻⁵ (h*Liter)⁻¹ for the fast and slow dosage forms, respectively. The mean plasma oxycodone concentration profiles after repeated dosing are shown in FIG. 15B. The steady state quartile AUC values for the formulations are set forth in Table 6.

The steady-state plasma profiles for both the q6h regimen of the IR product and the once daily regimen of the slow formulation were of the >18% quartile type while that for the once daily regimen of the fast formulation was not. Based on the findings of this study, the osmotic dosage form was changed to have 5% of the labeled dose in the overcoat to enable rapid dissolution and absorption after ingestion, and 95% of the labeled dose in the core for slow release over the entire dosing interval, i.e., 24 hours. This modified design was evaluated in a Phase I pharmacokinetic/pharmacodynamic study (Example 5) and in a Phase II dose-ranging study in osteoarthritis pain (Example 7).

### Example 5

### Pilot Study to Evaluate SZO-24 Oxycodone Hydrochloride Pharmacodynamics

A single-center, randomized, three-treatment, double-blind, crossover study was performed to compare the Example 2 SZO-24 dosage form (2×20 mg), IV morphine (10 mg), and placebo in healthy male subjects. This study was designed to determine the dose of oxycodone HCI when administered by the Example 2 SZO-24 dosage form that provides a statistically significant pharmacodynamic response as measured by the cold pain test.

Twelve male subjects enrolled and received all three treatments according to a randomly assigned sequence:
IV placebo and oral placebo;
IV morphine infusion (10 mg over 15 min) and oral placebo;
Example 2 SZO-24 dosage form (2×20 mg) and IV placebo (saline).

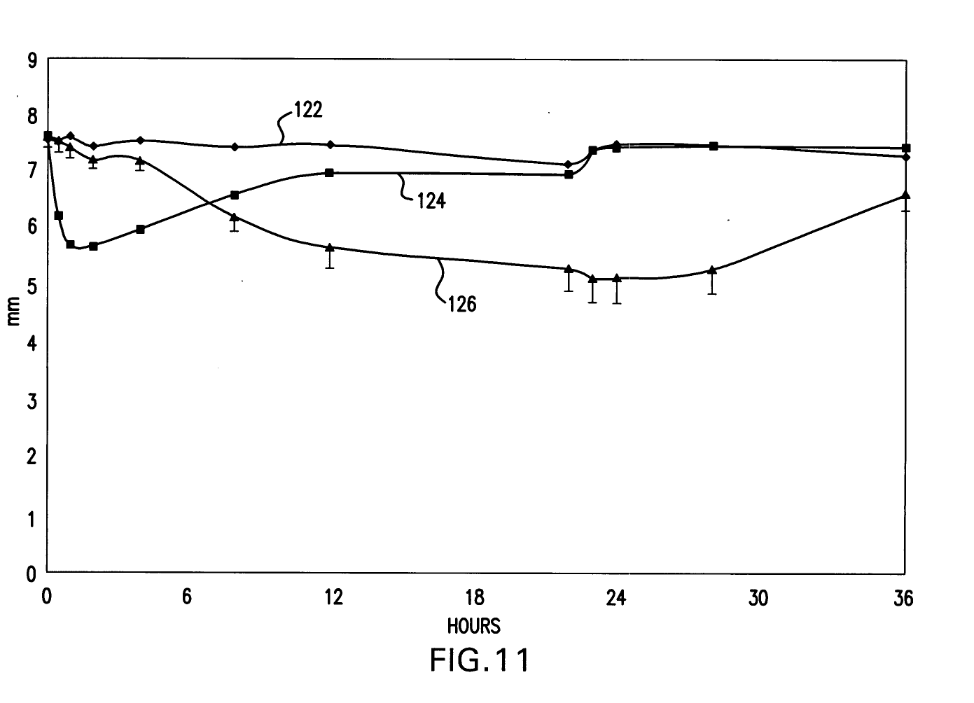

The treatment of IV morphine was intended to serve as a positive control due to the successful separation of this treatment from placebo as reported previously (Van and Rolan 1996), however, in this study, this treatment did not statistically separate from placebo as measured by the cold pain test. The pupil size remained steady over the study period for the placebo treatment, and the pupil size changes for both the IV morphine and the Example 2 SZO-24 dosage form were consistent with their respective pharmacokinetic profiles (see FIG. 11).

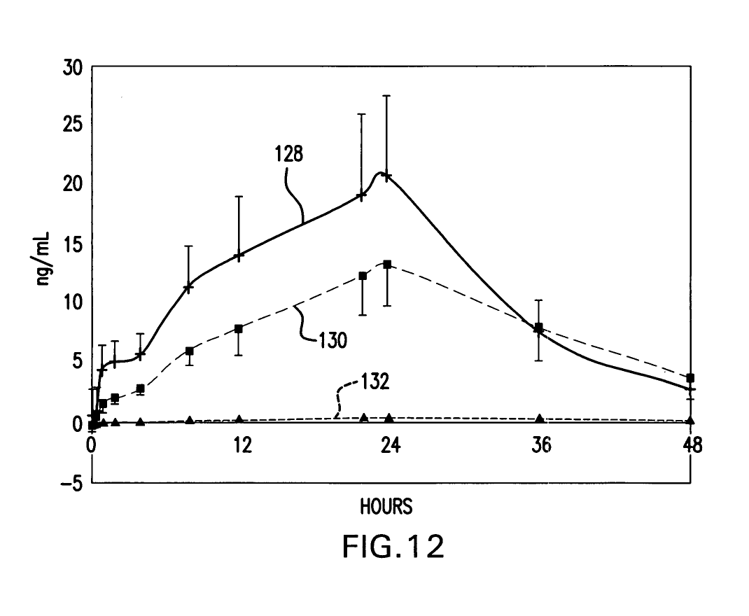

The study generated single-dose plasma oxycodone, noroxycodone, and oxymorphone concentration profiles for the Example 2 SZO-24 dosage form (2×20 mg) (see FIG. 12 and Table 7). The mean Cₘₐₓ/(Tₘₐₓ*Dose) ratio for oxycodone for this study was 2×10⁻⁵ (h*Liter)⁻¹.

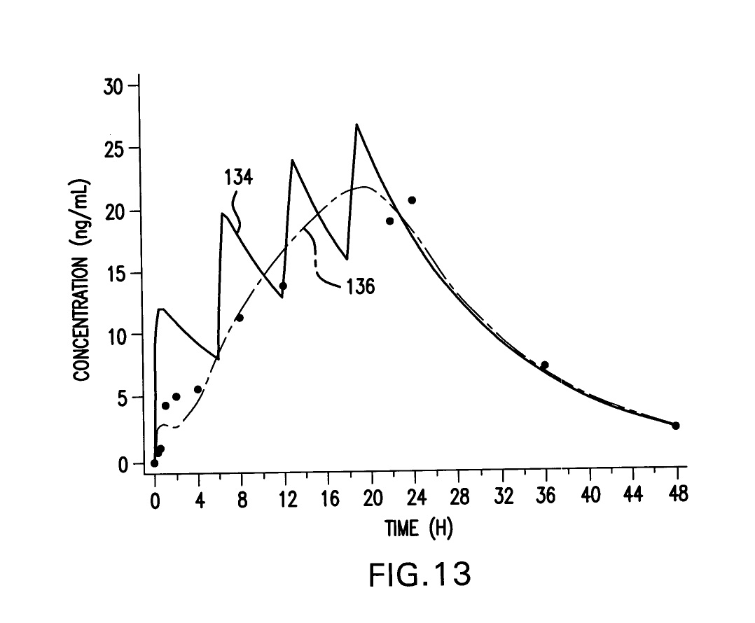

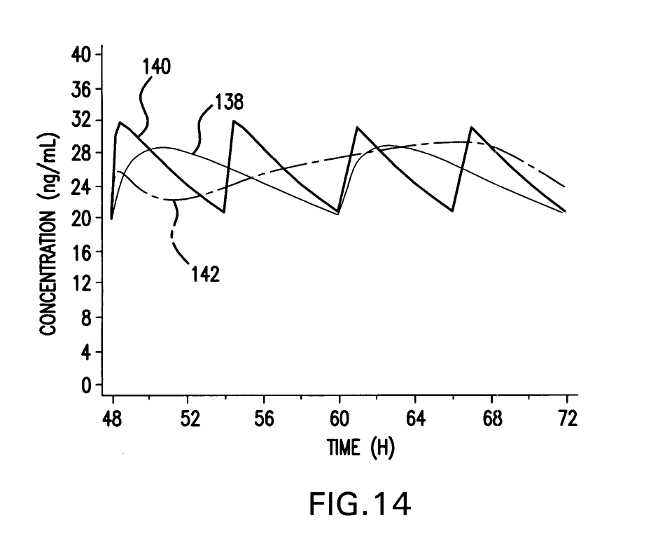

A pharmacokinetic model consisting of the in-vitro release rate for the Example 2 SZO-24 dosage form and a first-order absorption, first-order elimination disposition model was fitted to the plasma oxycodone concentration data using NONMEM. As the data were not sensitive to the absorption rate constant, the absorption rate constant was set to 6.48 h⁻¹. The population mean apparent clearance (Cl/F) was 67.7 L/h and the population mean apparent volume (V/F) was 556 L. The mean best-fit curve underestimated the mean data during the first few hours after dosing as shown in FIG. 13. The expected pharmacokinetic profile for IR oxycodone HCl, 10 mg, given every 6 hours was also simulated and is included in FIG. 13. The simulated steady-state pharmacokinetic profiles for a q6h regimen of an IR product, a q12h regimen of OXYCONTIN, and a qd regimen of the Example 2 SZO-24 dosage form are presented in FIG. 14. Based on the pharmacokinetic results, this formulation (5% of the labeled dose in the overcoat to enable rapid dissolution and absorption after ingestion, and 95% of the labeled dose in the core for slow release over the entire dosing interval, i.e., 24 hours, was further evaluated in a Phase II clinical study (Example 7).

### Example 6

### Single- and Multiple-Dose Pharmacokinetics of SZO-24 Oxycodone Hydrochloride and OXYCONTIN

This study was a single-center, randomized, open-label, two-treatment, two-period, single- and multiple-dose crossover study in healthy subjects. Subjects received the following treatments:
Treatment A—a single dose of the Example 3 SZO-24 dosage form (80 mg) followed 72 hours later by a QD regimen of the same dosage form (80 mg for 5 days);
Treatment B—two doses of OXYCONTIN®, 40 mg each dose, administered 12-hours apart followed 72 hours later by a q12h regimen of OXYCONTIN, 40 mg for 5 days.

All subjects took 50 mg naltrexone orally starting 14 hours before dosing and every 12 hours during the treatment periods and 24 hours after the last dosing day of oxycodone.

There was a minimum washout period of 5 days but not more than 14 days between treatment periods.

The objectives of the study were:
To determine the plasma oxycodone concentration profile for a single dose of the Example 3 SZO-24 dosage form (80 mg) and the steady-state plasma oxycodone hydrochloride concentration profile for a QD regimen of the dosage form;
To compare the steady-state plasma oxycodone concentration profile for a QD regimen of the Example 3 SZO-24 dosage form (80 mg), and that for a q12h regimen of OXYCONTIN.

A total of 37 subjects completed the study. The mean plasma oxycodone concentration profiles are given in FIG. 16. The mean plasma oxycodone concentration profile after the administration of the Example 3 SZO-24 dosage form (80 mg) can be found in FIG. 16B and the same profile is plotted with the mean profile after the administration of two OXYCONTIN (40 mg each) separated by 12 hours in FIG. 16C. From these figures and, in particular, the 12 hour data point for the Example 3 SZO-24 dosage form and the standard deviation for that data point, it can be seen that the single dose plasma profile for the dosage forms of the invention satisfies the relationship:
2.7×10⁻⁴ liter⁻¹≦C₁₂/D≦5.7×10⁻⁴ liter⁻¹.

For comparison, using the same 37 subjects, b.i.d. OXYCONTIN dosing was found to produce a mean C₁₂ concentration of 15.92 ng/ml (SD=6.88 ng/ml).

Dividing this mean value by 80mg, the total OXYCONTIN dose over 24 hours, gives 2.0×10⁻⁴ liters⁻¹, which is substantially below the above range for the once-a-day dosage forms of the invention.

The steady-state plasma concentration profiles for a once daily regimen of the Example 3 SZO-24 dosage form (80 mg) and twice daily dosing of OXYCONTIN, (40 mg each) can be found in FIG. 16D. PK data are summarized in Tables 8 (single dose) and 9 (steady state).

After the single administration of the Example 3 SZO-24 dosage form, the mean ratio of the area under the plasma concentration profile from 0 to 12 hour to AUC_{inf} was 0.24(0.07) and the mean ratio of the area under the plasma drug concentration profile from 12 to 24 hours to that from 0 to 12 hours was 1.94(0.49).

A comparison of plasma oxycodone concentrations at 72 (day 3), 96 (day 4), and 120 (day 5) hours following start of dosing during the multi-dose period showed that steady state had been reached by day 4 of dosing for both treatments.

A comparison of PK parameter AUC₉₆₋₁₂₀ on day 5 of the multi-dose period with AUC_{inf} following the single dose period for the Example 3 SZO-24 dosage form demonstrated time-invariant kinetics for this formulation (p=0.9).

The bioavailability for the Example 3 SZO-24 dosage form was 92% relative to OXYCONTIN as estimated by the AUC₉₆₋₁₂₀ ratio. The 90% confidence interval of this ratio falls within the 80-125% range for the bioequivalence criteria. Therefore, the amount of oxycodone provided by the Example 3 SZO-24 oxycodone dosage form given once-daily is bioequivalent to that of OXYCONTIN given twice-daily in the same total daily dose. The Cₘᵢₙ value for the Example 3 SZO-24 dosage form was 121% that for OXYCONTIN, while the Cₘₐₓ value for the Example 3 SZO-24 dosage form was 78% that for OXYCONTIN. The Cₘₐₓ values were significantly different (i.e., the ratio was significantly different from 1 (p<0.001)). These data demonstrate that the oxycodone plasma profile is flatter following treatment with the Example 3 SZO-24 dosage form as compared to treatment with OXYCONTIN.

The steady state quartile AUC values for the Example 3 SZO-24 dosage form and OXYCONTIN are set forth in Table 10. This data demonstrate that the Example 3 SZO-24 dosage form given once daily and OXYCONTIN given twice daily achieved >18%/quartile steady-state plasma oxycodone concentration profiles. Once daily dosing, however, is more convenient for patients and more likely to lead to better compliance. Also, as shown in FIG. 16D, the Example 3 SZO-24 dosage form produces a steady state profile that is clearly flatter than that produced by OXYCONTIN, which clearly continues to be biphasic.

### Example 7

### Phase II Clinical Study of SZO-24 Oxycodone Hydrochloride

A Phase II, two-week, placebo-controlled study using the Example 2 SZO-24 dosage form (20 mg and 2×20 mg=40 mg) in patients with osteoarthritis pain of the hips and/or knees was performed. In general, 40 mg showed statistically significant differences from placebo in pain assessments over the two-week treatment period, while 20 mg was superior to placebo over the first week of treatment but less consistently so during the second week, despite the fact that the study was not powered to show a statistically significant difference between the 20 mg and placebo in either week. The results showed the general trend that 40 mg was more effective than 20 mg, as expected, although the two dosage strengths did not show statistically different results in most cases. Scores of the Brief Pain Inventory (BPI), average pain intensity, showed significant results for both 20 mg (p=0.042) and 40 mg (p=0.010) at the last week on study medication.

Results from an analysis of the overall quality of sleep indicate that for the 40 mg treatment, the mean increases from baseline to last week of treatment and was statistically superior to placebo (p=0.0360) in improving the quality of sleep: 2.35 vs 1.21, on a scale of 0 (Very Poor) to 10 (Excellent).

### Example 8

### Rat Tolerance Study

This example reports the results of experiments performed to determine the effect of oxycodone input patterns on tolerance development in rats.

The specific objective of the study was to compare the degree of antinociceptive tolerance developed in rats administered oxycodone hydrochloride (HCl) for a period of three days, either by a biphasic dosing regimen (bolus/twice-a-day) or an SZO dosing regimen (substantially zero order/continuous). The biphasic dosing regimen used subcutaneous (SC) infusion, and the SZO regimen used subcutaneously-implanted ALZET® osmotic pumps. The vehicle control for the study was 0.9% saline. The test solutions were oxycodone HCl dissolved in saline.

The rodent tail-flick assay was used to assess analgesia (antinociception). This assay is a well-characterized and standard method to assess antinociception and tolerance to opioid drugs (Cleary 1994, D'Armour & Smith 1941). In the assay, rodents are briefly restrained and radiant heat is applied to the tip of the tail. The time it takes for the animal to flick its tail is recorded; a delay in this response compared to pre-dose readings is indicative of antinociception.

The tail flick latency methods used in the present study were similar to those described previously in the literature to assess antinociception (Duttaroy & Yoburn 1995, Nielsen et al 2000) with slight modifications from the original method described by D'Armour and Smith (1941). An IITC Model 33 Tail Flick Analgesia Meter was used to apply heat to the animal's tail (IITC Life Science, Woodland Hills, Calif.). The meter was programmed with the following conditions:
(1) Active Intensity: 75% (intensity of the stimulation light during the test);
(2) Trigger Temperature: 30° C. (this temperature allows pre-warming of the animal's tail to allow for more uniform measurements from day-to-day and test-to-test);
(3) Cutoff Time: 10 seconds (i.e., the length of time from the start of the test until the unit automatically ends the test to prevent tissue damage).

The animals were briefly restrained in plexiglass restrainers and radiant heat was applied to the tip of the animal's tail (approximately 1-2 cm from the tip). After the temperature reached 30° C., the meter increased the light intensity providing a noxious stimulus to the animal's tail. The time in seconds for the animal to flick its tail was recorded for each animal. If the animal did not flick its tail within 10 seconds (cutoff time), the heat stimulus was removed in order to minimize injury to the tail.

Three pre-dose readings were taken for each animal at intervals of approximately 5-15 minutes. For the animals used in the study, these pre-dose readings did not vary by more than a second for an individual animal. The average pre-dose readings for animals within the same test group did not vary by more than about two second (range=2.02 seconds). In this way, the variability of the measurements was decreased and thus the dynamic range of the assay was increased.

Tail flick latency values were converted to a percentage of the maximum possible effect (%MPE) using the following formula:
%MPE=100×(ΔL/ΔLₘₐₓ)
where:
ΔL=Post-dose Latency−Pre-dose Latency, and
ΔLₘₐₓ=Cutoff Time−Pre-dose Latency.

For the biphasic dosing regimen, the animals were subcutaneously infused using a computer-controlled Harvard syringe pump. The STANPUMP computer program (STANPUMP 1998) was used to drive an infusion device to administer test or control solutions as two boluses, approximately 12-hours apart. The animals had catheters implanted subcutaneously with approximately 7 cm of PE 10 tubing. The tubing was secured with sutures and sterile surgical skin glue to prevent accidental removal of the catheter. Prior to the start of infusion, the tubing was filled with the infusate (saline or oxycodone solutions).

During the treatment, the animals were connected to an Instech tethering system, which consisted of a Covance Infusion Harness and a stainless steel dual channel swivel mounted on a counter-balanced lever arm attached to an Instech MTANK cage. This tethering system allowed the rats to roam freely in their cages while protecting the catheters. The rat tethering system was designed to protect the surgically implanted catheters while providing free mobility to the rat during delivery. During infusion, the animals were housed singly and had free access to food and water. After approximately 72 hours of infusion, the tether system was disassembled, the suture was cut, and the catheter removed.

For each 24-hour period, the infusion regimen produced a biphasic profile, with two peaks (Cmax) between 2 to 4 hours and 14 and 16 hours, and two troughs (Cmin) at approximately 12 hours and 24 hours. The ratio of Cmax to Cmin was between three and four.

For the SZO dosing regimen, ALZET® osmotic pumps (Model 2ML1) were implanted subcutaneously in the animals. The pumps were primed overnight in 0.9% saline in an oven at 37° C. in order for the pump to have reached its steady state pumping rate at implantation (DURECT 2003). After approximately 72 hours, the pumps were removed. For the SZO dosing, the rats were not tethered.

Male Sprague-Dawley (SD) rats obtained from Charles River (Hollister, Calif.) and weighing at least 200g were used in the experiments. Extra animals were employed in the biphasic dosing regimen to take account of damaged catheters, but only enough animals were dosed on Day +3 to replace the animals with damaged catheters. The study was performed in compliance with the animal welfare regulations of 9 CFR 1-3 and the Guide for the Care and Use of Laboratory Animals (National Research Council 1996).

The animals were divided into four groups and on Day −1, each group was further divided into six subgroups and administered 0, 0.25, 0.5, 0.75, 1 or 1.5 mg/kg oxycodone by subcutaneous (SC) injection, respectively. Animals were tested for antinociception (tail-flick latency) approximately 15 minutes after injection. On Day 0, animals in each group were treated in accordance with Table 11.

After approximately 72 hours, the pumps infusing vehicle or oxycodone were stopped and the catheters were removed from the animals in Groups 1 and 2, and the ALZET® pumps were removed from the animals in Groups 3 and 4. Between six to eight hours after the end of infusion, each subgroup of Groups 1-4 was administered 0, 0.25, 0.5, 0.75, 1 or 3 mg/kg oxycodone by subcutaneous (SC) injection, respectively. Animals were tested for antinociception (tail-flick latency) approximately 15 minutes after injection. For both the biphasic and SZO dosing regimens, the dose of oxycodone over the 72 hour (3 day) test period was on average approximately 10 mg/kg·d, i.e., a total of approximately 30 mg/kg was administered over the testing period.

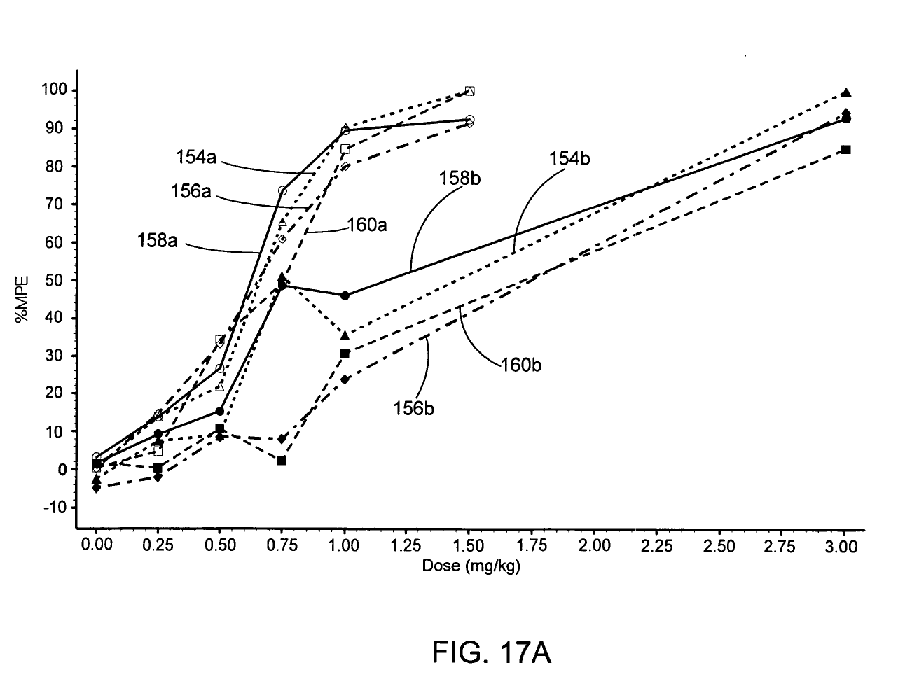

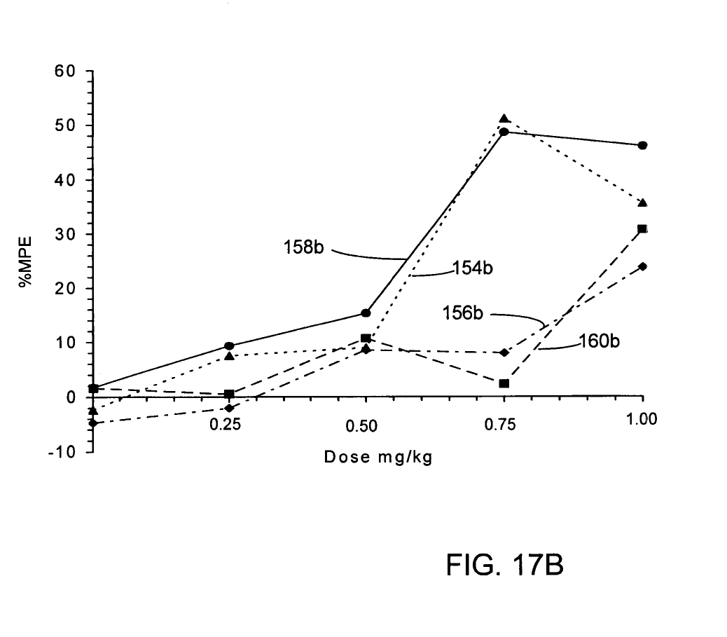

The results of these experiments are shown in Tables 12A and 12B, and in FIGS. 17A and 17B, where FIG. 17A plots all of the data of Tables 12A and 12B, while FIG. 17B plots the Day +3 data for tail flick testing doses of 0, 0.25, 0.5, 0.75, and 1.0 mg/kg. The curve numbers in FIGS. 17A and 17B correspond to the following:
curve 154a: SZO—Day −1/Saline Group;
curve 154b SZO—Day +3/Saline Group;
curve 156a: SZO—Day −1/Oxycodone Group;
curve 156b: SZO—Day +3/Oxycodone Group;
curve 158a: biphasic—Day −1/Saline Group;
curve 158b: biphasic—Day +3/Saline Group;
curve 160a: biphasic—Day −1/Oxycodone Group;
curve 160b: biphasic—Day +3/Oxycodone Group.

As can be seen most clearly in FIG. 17B, the groups that had been treated with oxycodone for 3 days (curves 156b and 160b) had generally smaller %MPE values for the same tail flick testing dose than the groups that had been treated with saline for 3 days (curves 154b and 158b), i.e., the oxycodone-treated animals had become tolerant to oxycodone so that the same tail flick testing dose generally had a smaller analgesic effect and thus produced less latency before a tail flick occurred.

Examination of the dose-effect curves suggests that not all the curves are likely to be modeled by the same equation. Also, the curves representing Day +3 data do not increase monotonically, and all four of the Day +3 effects at the 1 mg/kg test dose are below 50% of %MPE, thus making the estimation of ED50 difficult or with high uncertainty even with the much higher effect observed at 3 mg/kg.

Due to these modeling difficulties, an alternate approach was taken to obtain a statistical measure of the tolerance. The study design had each rat receiving the same testing dose of oxycodone on Day −1 and Day +3, except that the animals that received 1.5 mg/kg on Day −1, received 3.0 mg/kg on Day +3. Intuitively, the difference between the effect of the same test on Day +3 and Day −1 should be a direct measure of tolerance. The data collected from rats tested for responses at 0, 0.25, 0.5, 0.75, and 1 mg/kg were thus used to perform the statistical analysis.

For these rats, the overall study design followed a (2)×(2)×(5) format, i.e.:
(2) SZO dosing regimen versus biphasic dosing regimen
(2) oxycodone treatment versus saline treatment
(5) 0 mg/kg versus 0.25 mg/kg versus 0.5 mg/kg versus 0.75 mg/kg versus 1.0 mg/kg tail flick testing.

The total number of rats included in the analysis of the (2)×(2)×(5) format was 158. The data (difference between Day +3 and Day −1) were analyzed by the analysis of variance (ANOVA) method. The full variance model consisted of the three primary factors, their first-order interaction terms and their second order interaction term, namely:
dosing regimen,
3-day treatment,
tail flick testing dose,
dosing regimen×3-day treatment,
dosing regimen×tail flick testing dose,
3-day treatment×tail flick testing dose, and
dosing regiment×3-day treatment x tail flick testing dose.

The ANOVA analysis was performed with SAS software. None of the four interaction terms nor the dosing regimen term in the ANOVA model was statistically significant (with a critical α-value at 0.05). There was a statistically significant effect of the 3-day treatment (p=0.0039) and the tail flick testing dose (p<0.0001).

Therefore, the ANOVA analysis concluded that the tolerance was statistically different between rats treated with oxycodone for 3 days versus those treated with saline, and different between rats tested at different tail flick testing doses, but not statistically significantly different between rats treated with the SZO dosing regimen versus the biphasic dosing regimen.

Due to the lack of statistically significant interaction terms in the full ANOVA model, the data were further analyzed using a reduced ANOVA model containing only the primary design factors: dosing regiment, 3-day treatment, tail flick testing dose. This further analysis revealed the same conclusions as the analysis with the full ANOVA model. The tolerance was significantly different between oxycodone and saline treated rats (p=0.0035) and between rats tested with different doses of oxycodone (p<0.0001). The tolerance, however, was again not statistically significantly different between rats treated with the SZO dosing regimen versus the biphasic dosing regimen. The estimated mean tolerance difference was −10.7%MPE between the oxycodone and saline treated rats and −3.2%MPE between with the SZO dosing regimen and the biphasic dosing regimen. The −10.7%MPE difference was statistically different at an α-value of 0.05, but the −3.2% MPE value was not.

The lack of a statistically significantly difference between rats treated with a SZO dosing regimen versus a biphasic dosing regimen is in direct contrast with the concerns expressed in the literature that substantially zero order dosing will be more likely to lead to tolerance than biphasic dosing (see Benziger et al. 1997 and Kaiko 1997 discussed above). Based on this literature, one would have expected that the rats treated with the SZO dosing regimen would have exhibited more tolerance at a statistically significant level than those treated with the biphasic dosing regimen, but no such statistically significant difference was found.

From the foregoing, it can be seen that the invention provides dosage forms suitable for providing once-daily dosing of oxycodone and/or one or more its pharmaceutically-acceptable salts for relief of moderate to severe pain in patients requiring an opioid for more than a few days. Potential advantages for once-a-day dosing over current IR and CR oxycodone formulations include improved convenience, better compliance, a simpler dosing regimen, and more consistent pain relief with fewer adverse events over a 24-hour period.

Although specific embodiments of the invention have been described and illustrated, it is to be understood that a variety of modifications which do not depart from the scope and spirit of the invention will be evident to persons of ordinary skill in the art from the foregoing disclosure.

### References

Citations for various of the documents referred to above are set forth below. The contents of these documents, as well as those referenced elsewhere in this specification, are incorporated herein by reference.
Benzinger et al., “A Pharmacokinetic/Pharmacodynamic Study of Controlled-Release Oxycodone,” Journal of Pain and Symptom Management, 1997, 13:75-82
Cleary J, Mikus G, Somogyi A, Bochner F. The Influence of Pharmacogenetics on Opioid Analgesia: Studies with Codeine and Oxycodone in the Sprague-Dawley/dark Agouti Rat Model. J. Pharmacol Exp Ther 1994; 271:1528-1534
D'Armour F E, Smith D L. A Method for Determining Loss of Pain Sensation. J Pharmacol Exp Ther 1941; 72:74-79.
DURECT Corporation, 2003. ALZET Osmotic Pump Model 2ML1, Instruction and Specification Sheet.
Duttaroy A, Yoburn B C. The Effect of Intrinsic Efficacy on Opioid Tolerance. Anesthesiology 1995; 82;1226-1236.
Ekblom M, Hammarlund-Udenaes M, Paalzow L. Modeling of Tolerance development and rebound Effect During Different Intravenous Administrations of Morphine to Rats. J Pharmacol Exp Ther 1993; 266(1):244-252.
Gardmark M, Ekblom, M, Bouw R, Hammarlund-Udenaes M. Quantifiication of the Effects Delay and Acute Tolerance Development to Morphine in the Rat. J Pharmacol Exp Therap 1993; 267(5):1061-1067.
Kaiko R F. Pharmacokinetics and Pharmacodynamics of Controlled Release Opioids 1997 Acta Anaesthiol Scand 1997; 41:166-174
Nielsen C K, Ross F B, Smith M T. Incomplete, Asymmetric, and Route-Dependent Cross-Tolerance between Oxycodone and Morphine in the Dark Agouti Rat. J Pharmacol Exp Ther 2000; 295(1): 91-99.
Ouellet D M-C, Pollack G M. A Pharmacokinetic-Prarmacodynamic Model of Tolerance to Morphine Analgesia During Infusion in Rats. J. Pharmacokinetics Biopharmaceutics. 1995; 23(6):531-549
Ouellet D M-C, Pollack G M. Pharmacodynamics and Tolerance Development During Multiple Intravenous Bolus Morphine Administration in Rats. J Pharmacol Exp Ther 1997; 281(2):713-720.
Van, F. and P. E. Rolan. The utility of the cold pain test to measure analgesia from intravenous morphine. Br J Clin Pharmacol. 1996; 42: 663-4.
STANPUMP User's Manual 1998 http ://anesthesia.stanford.edu/pkpd/Target%20Control%20Drug%20Delivery/STANPUMP/Forms/AllItems.htm (August 2004)

National Research Council. Guide for the Care and Use of Laboratory Animals. Washington D.C.: National Academy Press 1996.

TABLE 1

| | Mean Value | | |
|---|---|---|---|
| | Mandema et al. | | |
| Parameter | 1996 | Example 5 | Example 6 |
| CL/F (l h⁻¹) | 110 | 67.7 | 80 |
| Vd/F (l) | 593 | 556 | 431 |
| ka (h⁻¹) | 4.19 | 6.48 | 4.19 |
| ke (h⁻¹) | 0.186 | 0.122 | 0.186 |

TABLE 2

Release
Rate (mg/h)

| | 0-12 h | 12-24 h | Cmax (ng/mL) |
|---|---|---|---|
| Fast-Slow | 5 | 1.67 | 56.4 |
| Slow-Fast | 1.67 | 5 | 60.9 |

TABLE 3

| | | |
|---|---|---|
| Material | % | mg |

Push Granulation

| | | |
|---|---|---|
| Polyethylene Oxide, NF, 7000K, TG | 73.73% | 75.94 |
| Povidone, USP, Ph Eur, (K29-32) | 5.00% | 5.15 |
| Sodium Chloride, USP, Ph Eur, (Powder) | 20.00% | 20.6 |
| Magnesium Stearate, NF, Ph Eur | 0.25% | 0.26 |
| BHT, FCC, Ph Eur, (Milled) | 0.02% | 0.02 |
| Iron Oxide, Green PB-1581 | 1.00% | 1.03 |

Active Granulations

| | | |
|---|---|---|
| Oxycodone Hydrochloride, USP | 15.80% | 17.00 |
| Polyethylene Oxide N80, TG LEO | 81.68% | 92.30 |
| Povidone, USP, Ph Eur, (K29-32) | 2.00% | 2.26 |
| Magnesium Stearate, NF, Ph Eur | 0.50% | 0.57 |
| BHT, FCC, Ph Eur, (Milled) | 0.02% | 0.02 |

Membrane Coat:

| | | fast | slow |
|---|---|---|---|
| Material | % | mg | mg |
| Cellulose Acetate, NF, (398-10) | 4.95% | 19.80 | 29.70 |
| Polyethylene Glycol 3350, NF, LEO | 0.05% | 0.20 | 0.30 |
| Acetone, NF, (Bulk) | 90.25% | — | — |
| Purified Water, USP, Ph Eur | 4.75% | — | — |

| Unit Weights: | fast | | slow |
|---|---|---|---|
| Drug Layer Weight | (mg) | 113 | 113 |
| Push Layer Weight | (mg) | 103 | 103 |
| Membrane Coating Weight | (mg) | 20 | 30 |

TABLE 4

| | | |
|---|---|---|
| Material | % | mg |

Push Granulation

| | | |
|---|---|---|
| Polyethylene Oxide, NF, 7000K, TG | 73.73% | 75.94 |
| Povidone, USP, Ph Eur, (K29-32) | 5.00% | 5.15 |
| Sodium Chloride, USP, Ph Eur, (Powder) | 20.00% | 20.6 |
| Magnesium Stearate, NF, Ph Eur | 0.25% | 0.26 |
| BHT, FCC, Ph Eur, (Milled) | 0.02% | 0.02 |
| Iron Oxide, Green PB-1581 | 1.00% | 1.03 |

Active Granulations:

| | | |
|---|---|---|
| Oxycodone Hydrochloride, USP | 17.70% | 20.00 |
| Polyethylene Oxide N80, TG LEO | 78.03% | 88.17 |
| Povidone, USP, Ph Eur, (K29-32) | 4.00% | 4.52 |
| Magnesium Stearate, NF, Ph Eur | 0.25% | 0.28 |
| BHT, FCC, Ph Eur, (Milled) | 0.02% | 0.02 |

Membrane Coat:

| | | |
|---|---|---|
| Cellulose Acetate, NF, (398-10) | 4.95% | 37.62 |
| Polyethylene Glycol 3350, NF, LEO | 0.05% | 0.38 |
| Acetone, NF, (Bulk) | 90.25% | — |
| Purified Water, USP, Ph Eur | 4.75% | — |

Drug Coat:

| | | |
|---|---|---|
| Oxycodone Hydrochloride, USP | 1.50% | 1 |
| HPMC 2910, USP, Ph Eur, 3cps | 8.50% | 6 |
| Purified Water, USP, Ph Eur | 90.00% | — |

Color Coat:

| | | |
|---|---|---|
| Opadry ®, Gray (TS-009525) | 12.00% | 8 |
| Purified Water, USP, Ph Eur | 88.00% | — |

Clear Coat:

| | | |
|---|---|---|
| Opadry ®, Clear (YS-1-19025-A) | 5.00% | 3.2 |
| Purified Water, USP, Ph Eur | 95.00% | — |
| Carnauba Wax, NF, (Powder) | 0.01% | 0.05 |

| Unit Weights: | | 20 mg |
|---|---|---|
| Drug Layer Weight | (mg) | 113 |
| Push Layer Weight | (mg) | 103 |
| Membrane Coating Weight | (mg) | 38 |
| Drug Overcoat Weight | (mg) | 7 |
| Color Overcoat Weight | (mg) | 8 |
| Clear Overcoat Weight | (mg) | 3.2 |

TABLE 5

| | | |
|---|---|---|
| Material | % | mg |

Push Granulation:

| | | |
|---|---|---|
| Polyethylene Oxide, NF, 7000K, TG, LEO | 73.73% | 141.56 |
| Povidone, USP, Ph Eur, (K29-32) | 5.00% | 9.60 |
| Sodium Chloride, USP, Ph Eur, (Powder) | 20.00% | 38.40 |
| Magnesium Stearate, NF, Ph Eur | 0.25% | 0.48 |
| BHT, FCC, Ph Eur, (Milled) | 0.02% | 0.04 |
| Iron Oxide, Green PB-1581 | 1.00% | 1.92 |

Active Granulation:

| | | |
|---|---|---|
| Oxycodone Hydrochloride, USP | 32.00% | 80.00 |
| Polyethylene Oxide N150 FP LEO | 63.71% | 159.28 |
| Povidone, USP, Ph Eur, (K29-32) | 4.00% | 10.00 |
| Ferric Oxide, NF, (Red) | 0.02% | 0.05 |
| Magnesium Stearate, NF, Ph Eur | 0.25% | 0.63 |
| BHT, FCC, Ph Eur, (Milled) | 0.02% | 0.05 |

Membrane Coat:

| | | |
|---|---|---|
| Cellulose Acetate, NF, (398-10) | 4.95% | 43.56 |
| PEG 3350 | 0.05% | 0.44 |
| Acetone, NF, (Bulk) | 90.25% | |
| Purified Water, USP, Ph Eur | 4.75% | |

Drug Coat:

| | | |
|---|---|---|
| Oxycodone Hydrochloride, USP* | 1.80% | 4.00 |
| Opadry Clear YS-1-19025-A | 10.20% | 22.67 |
| Purified Water, USP, Ph Eur | 88.00% | — |

Color Coat:

| | | |
|---|---|---|
| Opadry ®, Red (No. 03B15632) | 12.00% | 8.00 |
| Carnauba Wax, NF, (Powder) | 0.01% | trace |

| | | 80 mg |
|---|---|---|
| Unit Weights: | | |
| Drug Layer Weight | (mg) | 250 |
| Push Layer Weight | (mg) | 192 |
| Membrane Coating Weight | (mg) | 44 |
| Drug Overcoat Weight* | (mg) | 26.7 |
| Color Overcoat Weight | (mg) | 8 |
| Formulation Characteristics: | | |
| Tablet Size | (in) | 13/32″ |
| Core content* | (mg) | 80 |
| Drug overcoat content | (mg) | 4 |
| Total drug content | (mg) | 84 |

*including 5% system overage in core

TABLE 6
Mean (SD) Ratio of AUC For Each Quartile
to AUC For the Entire (0-24 hr) Steady-State Profile

| | 0-6 h | 6-12 h | 12-18 h | 18-24 h |
|---|---|---|---|---|
| IR 5 mg q6h | 0.29(0.03) | 0.27(0.03) | 0.19(0.03) | 0.24(0.03) |
| Fast | 0.19(0.03) | 0.36(0.05) | 0.29(0.03) | 0.16(0.03) |
| Slow | 0.20(0.03) | 0.28(0.03) | 0.30(0.02) | 0.23(0.04) |

TABLE 7
Single-Dose Plasma Concentrations for
40 mg (Oxycodone HCl) SZO-24 Dosage Form

| | Cmax (ng/mL) | AUCinf (h × ng/mL) |
|---|---|---|
| Oxycodone | 20.92 | 553.2 |
| Noroxycodone | 13.12 | 421.2 |
| Oxymorphone | 0.35 | 11.67 |

TABLE 8
Mean (SD) Oxycodone PK Parameters Following Single Dose

| | SZO-24 Oxycodone | OXYCONTIN |
|---|---|---|
| | (80 mg) | 40 mg q12h |
| Cₘₐₓ (ng/mL) | 41.2 (13.1) | 57.5 (18.6) |
| Tₘₐₓ (h) | 19.4 (5.1) | 15.1 (4.4) |
| Cₘₐₓ/(Tₘₐₓ × Dose) | 4 × 10⁻⁵ (2 × 10⁻⁵) | 4 × 10⁻⁴ (3 × 10⁻⁴)^{a} |
| (h × Liter)⁻¹ | | |
| t_{1/2} (h) | 5.4 (0.9) | 5.1 (0.6) |
| AUC₀₋₄₈ (ng/mL · h) | 971.4 (361.7) | 1007.3 (330.2) |
| AUC_{inf} (ng/mL · h) | 989.2 (376.1) | Not done |

^{a}This calculation used Cmax and Tmax during the first dosing interval (0 to 12 hr).

TABLE 9
Mean (SD) Oxycodone PK Parameters Following Multi-Dose

| | SZO-24 Oxycodone | OXYCONTIN |
|---|---|---|
| | (80 mg) | 40 mg q12h |
| Cₘₐₓ (ng/mL) | 53.2 (15.3) | 67.3 (19.5) |
| Tₘₐₓ (h) | 105.1 (8.6) | 104.8 (6.6) |
| Cₘᵢₙ (ng/mL) | 29.3 (12.8) | 21.0 (7.9) |
| Tₘᵢₙ (h) | 109.3 (9.5) | 106.6 (7.1) |
| AUC₉₆₋₁₂₀ (ng/mL · h) | 988.9 (296.3) | 1063.7 (338.0) |

TABLE 10
Mean (SD) Ratio of AUC for Each Quartile
to AUC for the Entire (0-24 hr) Steady-State Profile

| | 0-6 h | 6-12 h | 12-18 h | 18-24 h |
|---|---|---|---|---|
| SZO-24 Oxycodone | 0.27(0.08) | 0.26(0.04) | 0.24(0.05) | 0.23(0.06) |
| OXYCONTIN | 0.30(0.02) | 0.19(0.02) | 0.29(0.03) | 0.22(0.03) |

TABLE 11

| Group | | | Oxycodone Dose | Number of |
|---|---|---|---|---|
| Number | Treatment | Route | mg/(kg · d)^{a} | Animals |
| 1 | Vehicle^{b} | SC Infusion | 0 | 47 |
| | | (syringe pump) | | |
| 2 | Oxycodone | SC Infusion | 10 | 47 |
| | | (syringe pump) | | |
| 3 | Vehicle^{b} | SC ALZET | 0 | 48 |
| 4 | Oxycodone | SC ALZET | 10 | 48 |

^{a}Doses calculated in terms of the hydrochloride salt.
^{b}0.9% saline.

TABLE 12A
SZO Dosing

| | Tail Flick | Results¹ | |
|---|---|---|---|
| | Testing Dose | (% MPE) | |
| Treatment | (mg/kg) | Day −1 | Day +3 |
| Saline | 0 | 0.50 ± 6.20 | −2.37 ± 7.58 |
| Saline | 0.25 | 13.69 ± 8.94 | 7.44 ± 11.94 |
| Saline | 0.5 | 21.86 ± 13.71 | 8.92 ± 11.30 |
| Saline | 0.75 | 65.33 ± 32.06 | 51.09 ± 46.28 |
| Saline | 1 | 90.33 ± 19.58 | 35.55 ± 27.94 |
| Saline | 1.5² | 100.00 ± 0.00 | 100.00 ± 0.00 |
| Oxycodone | 0 | 0.33 ± 5.14 | −4.75 ± 8.51 |
| Oxycodone | 0.25 | 14.72 ± 16.16 | −2.07 ± 4.18 |
| Oxycodone | 0.5 | 33.12 ± 19.58 | 8.48 ± 17.38 |
| Oxycodone | 0.75 | 60.76 ± 31.45 | 7.90 ± 11.36 |
| Oxycodone | 1 | 80.15 ± 36.76 | 23.75 ± 25.96 |
| Oxycodone | 1.5² | 91.44 ± 24.22 | 94.35 ± 11.48 |

¹mean ± SD; n = 8.
²3.0 mg/kg for Day +3.

TABLE 12B
Biphasic Dosing

| | Tail Flick | Results¹ | |
|---|---|---|---|
| | Testing Dose | (% MPE) | |
| Treatment | (mg/kg) | Day −1 | Day +3 |
| Saline | 0 | 3.20 ± 7.23 | 1.71 ± 4.11 |
| Saline | 0.25 | 13.82 ± 7.87 | 9.25 ± 12.00 |
| Saline | 0.5 | 26.59 ± 22.79 | 15.22 ± 12.44 |
| Saline | 0.75 | 73.56 ± 28.57 | 48.59 ± 39.89 |
| Saline³ | 1 | 89.52 ± 20.25 | 46.00 ± 18.96 |
| Saline | 1.5² | 92.48 ± 16.05 | 92.97 ± 15.65 |
| Oxycodone³ | 0 | 0.47 ± 1.06 | 1.55 ± 4.70 |
| Oxycodone | 0.25 | 4.75 ± 6.80 | 0.48 ± 2.28 |
| Oxycodone | 0.5 | 34.19 ± 28.28 | 10.64 ± 7.72 |
| Oxycodone | 0.75 | 49.32 ± 32.89 | 2.26 ± 8.35 |
| Oxycodone | 1 | 84.61 ± 27.43 | 30.73 ± 19.05 |
| Oxycodone | 1.5² | 100.00 ± 0.00 | 84.81 ± 26.06 |

¹mean ± SD; n = 8, except where indicated.
²3.0 mg/kg for Day +3.
³n = 7.

1. A controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said formulation providing (a) a mean, single dose, maximum plasma concentration Cₘₐₓ and (b) a mean, single dose, area under a plasma concentration-time curve for 0-48 hours AUC₀₋₄₈ which satisfy the relationships:
3.5×10⁻⁴ liter⁻¹≦Cₘₐₓ/D≦6.8×10⁻⁴ liter⁻¹, and
7.6×10⁻³ hour/liter≦AUC₀₋₄₈/D≦16.7×10⁻³ hour/liter,
wherein said formulation provides pain relief for about 24 hours or more after administration to the patient.

2. The formulation of claim 1 wherein Cₘₐₓ and AUC₀₋₄₈ are determined using plasma samples from individuals to whom one or more opioid antagonists have been administered.

3. The formulation of claim 1 wherein Cₘₐₓ and AUC₀₋₄₈ are determined using plasma samples from individuals to whom naltrexone has been administered.

4. The formulation of claim 1 wherein Cₘₐₓ and AUC₀₋₄₈ are determined using plasma samples from individuals who have not been administered an opioid antagonist.

5. The formulation of claim 1 wherein Cₘₐₓ and AUC₀₋₄₈ are determined using plasma samples from individuals who have not been administered naltrexone.

6. The formulation of claim 1, 2, or 4 wherein said formulation provides a mean, single dose, time to maximum plasma concentration Tₘₐₓ which satisfies the relationship:
Tₘₐₓ>17 hours:

7. The formulation of claim 6 wherein Tₘₐₓ satisfies the relationship:
Tₘₐₓ>18 hours.

8. The formulation of claim 6 wherein Tₘₐₓ satisfies the relationship:
Tₘₐₓ>19 hours.

9. The formulation of claim 1, 2, or 4 wherein said formulation provides a mean, single dose, time to maximum plasma concentration Tₘₐₓ, and D, Cₘₐₓ, and Tₘₐₓ satisfy the relationship:
Cₘₐₓ/(Tₘₐₓ·D)≦3×10⁻⁴ (liter·hour)⁻¹.

10. The formulation of claim 9 wherein D, Cₘₐₓ, and Tₘₐₓ satisfy the relationship:
2×10⁻⁵ (liter·hour)⁻¹≦Cₘₐₓ/(Tₘₐₓ·D)≦6×10⁻⁵ (liter·hour)⁻.

11. The formulation of claim 1, 2, or 4 wherein said formulation provides mean, single dose, areas under a plasma concentration-time curve for 0-12 hours AUC₀₋₁₂ and for 12-24 hours AUC₁₂₋₂₄ which satisfy the relationship:
AUC₁₂₋₂₄/AUC₀₋₁₂>1.0.

12. The formulation of claim 11 wherein AUC₀₋₁₂ and AUC₁₂₋₂₄ satisfy the relationship:
AUC₁₂₋₂₄/AUC₀₋₁₂>1.5.

13. The formulation of claim 11 wherein AUC₀₋₁₂ and AUC₁₂₋₂₄ satisfy the relationship:
AUC₁₂₋₂₄/AUC₀₋₁₂>1.7.

14. The formulation of claim 11 wherein AUC₀₋₁₂ and AUC₁₂₋₂₄ satisfy the relationship:
AUC₁₂₋₂₄/AUC₀₋₁₂>2.0.

15. The formulation of claim 1, 2, or 4 wherein:
(a) the dose comprises a first component for immediate release and a second component for sustained release; and
(b) the weight ratio W of the first component to the sum of the first and second components is less than about 0.25.

16. The formulation of claim 15 where D is about 20 mg and W is about 0.05.

17. A controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
wherein:
(a) the formulation provides a mean, single dose, plasma concentration profile that increases substantially monotonically over 24 hours or more;
(b) the formulation provides a mean, single dose, area under a plasma concentration-time curve for 0-48 hours AUC₀₋₄₈ which satisfies the relationship:
7.6×10⁻³ hour/liter≦AUC₀₋₄₈/D<16.7×10⁻³ hour/liter; and
(c) the formulation provides pain relief for about 24 hours or more after administration to the patient.

18. The formulation of claim 17 wherein AUC₀₋₄₈ and the mean, single dose, plasma concentration profile are determined using plasma samples from individuals to whom one or more opioid antagonists have been administered.

19. The formulation of claim 17 wherein AUC₀₋₄₈ and the mean, single dose, plasma concentration profile are determined using plasma samples from individuals to whom naltrexone has been administered.

20. The formulation of claim 17 wherein AUC₀₋₄₈ and the mean, single dose, plasma concentration profile are determined using plasma samples from individuals who have not been administered an opioid antagonist.

21. The formulation of claim 17 wherein AUC₀₋₄₈ and the mean, single dose, plasma concentration profile are determined using plasma samples from individuals who have not been administered naltrexone.

22. The formulation of claim 17, 18, or 20 wherein the mean, single dose, plasma concentration profile comprises a first rising phase and a second phase, where the slope of the first rising phase is greater than the magnitude of the slope of the second phase.

23. The formulation of claim 22 wherein the transition between the first rising phase and the second phase occurs between 12 and 16 hours.

24. The formulation of claim 23 wherein the first rising phase comprises a first subphase and a second subphase, where the first subphase rises faster than the second subphase.

25. The formulation of claim 24 wherein the transition between the first subphase and the second subphase occurs between 1 and 3 hours.

26. The formulation of claim 17, 18, or 20 wherein:
(a) the dose comprises a first component for immediate release and a second component for sustained release; and
(b) the weight ratio W of the first component to the sum of the first and second components is less than about 0.25.

27. The formulation of claim 26 where D is about 20 mg and W is about 0.05.

28. A controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said formulation providing (a) a mean, single dose, 12 hour plasma concentration C₁₂ and (b) a mean, single dose, area under a plasma concentration-time curve for 0-48 hours AUC₀₋₄₈ which satisfy the relationships:
2.7×10⁻⁴ liter⁻¹<C₁₂/D≦5.7×10⁻⁴ liter⁻¹, and
7.6×10⁻³ hour/liter≦AUC₀₋₄₈/D≦16.7×10⁻³ hour/liter,
wherein said formulation provides pain relief for about 24 hours or more after administration to the patient.

29. The formulation of claim 28 wherein C₁₂ and AUC₀₋₄₈ are determined using plasma samples from individuals to whom one or more opioid antagonists have been administered.

30. The formulation of claim 28 wherein C₁₂ and AUC₀₋₄₈ are determined using plasma samples from individuals to whom naltrexone has been administered.

31. The formulation of claim 28 wherein C₁₂ and AUC₀₋₄₈ are determined using plasma samples from individuals who have not been administered an opioid antagonist.

32. The formulation of claim 28 wherein C₁₂ and AUC₀₋₄₈ are determined using plasma samples from individuals who have not been administered naltrexone.

33. The formulation of claim 28, 29, or 31 wherein:
(a) the dose comprises a first component for immediate release and a second component for sustained release; and
(b) the weight ratio W of the first component to the sum of the first and second components is less than about 0.25.

34. The formulation of claim 33 where D is about 20 mg and W is about 0.05.

35. A controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said formulation providing mean, steady state, areas under a plasma concentration-time curve for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ which satisfy the relationships:
AUC₀₋₆/AUC₀₋₂₄>0.18,
AUC₆₋₁₂/AUC₀₋₂₄>0.18,
AUC₁₂₋₁₈/AUC₀₋₂₄>0.18, and
AUC₁₈₋₂₄/AUC₀₋₂₄>0.18,
wherein said formulation provides pain relief for about 24 hours or more after administration to the patient.

36. The formulation of claim 35 wherein AUC₀₋₆, AUC₆₋₁₂, AUC₁₂₋₁₈, AUC₁₈₋₂₄ and AUC₀₋₂₄ are determined using plasma samples from individuals to whom one or more opioid antagonists have been administered.

37. The formulation of claim 35 wherein AUC₀₋₆, AUC₆₋₁₂, AUC₁₂₋₁₈, AUC₁₈₋₂₄ and AUC₀₋₂₄ are determined using plasma samples from individuals to whom naltrexone has been administered.

38. The formulation of claim 35 wherein AUC₀₋₆, AUC₆₋₁₂, AUC₁₂₋₁₈, AUC₁₈₋₂₄ and AUC₀₋₂₄ are determined using plasma samples from individuals who have not been administered an opioid antagonist.

39. The formulation of claim 35 wherein AUC₀₋₆, AUC₆₋₁₂, AUC₁₂₋₁₈, AUC₁₈₋₂₄ and AUC₀₋₂₄ are determined using plasma samples from individuals who have not been administered naltrexone.

40. The formulation of claim 35, 36, or 38 wherein AUC₀₋₆, AUC₆₋₁₂, AUC₁₂₋₁₈, AUC₁₈₋₂₄, and AUC₀₋₂₄ satisfy the relationships:
AUC₀₋₆/AUC₀₋₂₄>0.20,
AUC₆₋₁₂/AUC₀₋₂₄>0.20,
AUC₁₂₋₁₈/AUC₀₋₂₄>0.20, and
AUC₁₈₋₂₄/AUC₀₋₂₄>0.20.

41. The formulation of claim 35, 36, or 38 wherein the magnitude of the difference between any two of AUC₀₋₆/AUC₀₋₂₄, AUC₆₋₁₂/AUC₀₋₂₄, AUC₁₂₋₁₈/AUC₀₋₂₄, and AUC₁₈₋₂₄/AUC₀₋₂₄ is less than or equal to 0.05.

42. The formulation of claim 41 wherein the magnitude of the difference between each of:
AUC₀₋₆/AUC₀₋₂₄ and AUC₆₋₁₂/AUC₀₋₂₄,
AUC₆₋₁₂/AUC₀₋₂₄ and AUC₁₂₋₁₈/AUC₀₋₂₄,
AUC₁₂₋₁₈/AUC₀₋₂₄ and AUC18-24/AUC₀₋₂₄, and
AUC₁₈₋₂₄/AUC₀₋₂₄ and AUC₀₋₆/AUC₀₋₂₄
is less than or equal to 0.03.

43. The formulation of claim 35, 36, or 38 wherein the magnitude of the difference between each of:
AUC₀₋₆/AUC₀₋₂₄ and AUC₆₋₁₂/AUC₀₋₂₄,
AUC₆₋₁₂/AUC₀₋₂₄ and AUC₁₂₋₁₈/AUC₀₋₂₄,
AUC₁₂₋₁₈/AUC₀₋₂₄ and AUC₁₈₋₂₄/AUC₀₋₂₄, and
AUC₁₈₋₂₄/AUC₀₋₂₄ and AUC₀₋₆/AUC₀₋₂₄
is less than or equal to 0.03.

44. The formulation of claim 35, 36, or 38 wherein:
(a) the dose comprises a first component for immediate release and a second component for sustained release; and
(b) the weight ratio W of the first component to the sum of the first and second components is less than about 0.25.

45. The formulation of claim 44 where D is about 20 mg and W is about 0.05.

46. A controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said formulation having an in vitro release profile in which:
(a) 0-20% of the dose is released in 0-2 hours;
(b) 30-65% of the dose is released in 0-12 hours; and
(c) 80-100% of the dose is released in 0-24 hours;
wherein the release profile is determined using a USP Type VII bath indexer in a constant temperature water bath at 37° C. and wherein said formulation provides pain relief for about 24 hours or more after administration to the patient.

47. The formulation of claim 46 wherein 33-63% of the dose is released in 0-12 hours.

48. The formulation of claim 46 wherein:
(a) the dose comprises a first component for immediate release and a second component for sustained release; and
(b) the weight ratio W of the first component to the sum of the first and second components is less than about 0.25.

49. The formulation of claim 48 where D is about 20 mg and W is about 0.05.

50. A controlled-release oxycodone formulation for once-a-day oral administration to human patients comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
wherein:
(a) the dose comprises a first component for immediate release and a second component for sustained release; and
(b) the weight ratio W of the first component to the sum of the first and second components is less than about 0.25.

51. The formulation of claim 50 wherein W is less than about 0.10.

52. The formulation of claim 50 wherein W is less than or equal to about 0.05.

53. The formulation of claim 50 where D is about 20 mg and W is about 0.05.

54. A method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said dosage form providing (a) a mean, single dose, maximum plasma concentration Cₘₐₓ and (b) a mean, single dose, area under a plasma concentration-time curve for 0-48 hours AUC₀₋₄₈ which satisfy the relationships:
3.5×10⁻⁴ liter⁻≦Cₘₐₓ/D≦6.8×10⁻⁴ liter⁻¹, and
7.6×10⁻³ hour/liter≦AUC₀₋₄₈/D≦16.7×10⁻³ hour/liter,
wherein the dosage form provides pain relief for about 24 hours or more after administration to the patient.

55. The method of claim 54 wherein Cₘₐₓ and AUC₀₋₄₈ are determined using plasma samples from individuals to whom one or more opioid antagonists have been administered.

56. The method of claim 54 wherein Cₘₐₓ and AUC₀₋₄₈ are determined using plasma samples from individuals to whom naltrexone has been administered.

57. The method of claim 54 wherein Cₘₐₓ and AUC₀₋₄₈ are determined using plasma samples from individuals who have not been administered an opioid antagonist.

58. The method of claim 54 wherein Cₘₐₓ and AUC₀₋₄₈ are determined using plasma samples from individuals who have not been administered naltrexone.

59. The method of claim 54, 55, or 57 wherein the dosage form provides a mean, single dose, time to maximum plasma concentration Tₘₐₓ which satisfies the relationship:
Tₘₐₓ>17 hours.

60. The method of claim 59 wherein Tₘₐₓ satisfies the relationship:
Tₘₐₓ>18 hours.

61. The method of claim 59 wherein Tₘₐₓ satisfies the relationship:
Tₘₐₓ>19 hours.

62. The method of claim 54, 55, or 57 wherein the dosage form provides a mean, single dose, time to maximum plasma concentration Tₘₐₓ, and D, Cₘₐₓ, and Tₘₐₓ satisfy the relationship:
Cₘₐₓ/(Tₘₐₓ·D)≦3×10⁻⁴ (liter·hour)⁻¹.

63. The method of claim 62 wherein D, Cₘₐₓ, and Tₘₐₓ satisfy the relationship:
2×10⁻⁵ (liter·hour)⁻¹≦Cₘₐₓ/(Tₘₐₓ·D)≦6×10⁻⁵ (liter·hour)⁻¹.

64. The method of claim 54, 55, or 57 wherein the dosage form provides mean, single dose, areas under a plasma concentration-time curve for 0-12 hours AUC₀₋₁₂ and for 12-24 hours AUC₁₂₋₂₄ which satisfy the relationship:
AUC₁₂₋₂₄/AUC₀₋₁₂>1.0.

65. The method of claim 64 wherein AUC₀₋₁₂ and AUC₁₂₋₂₄ satisfy the relationship:
AUC₁₂₋₂₄/AUC₀₋₁₂>1.5.

66. The method of claim 64 wherein AUC₀₋₁₂ and AUC₁₂₋₂₄ satisfy the relationship:
AUC₁₂₋₂₄/AUC₀₋₁₂>1.7.

67. The method of claim 64 wherein AUC₀₋₁₂ and AUC₁₂₋₂₄ satisfy the relationship:
AUC₁₂₋₂₄/AUC₀₋₁₂>2.0.

68. The method of claim 54, 55, or 57 wherein:
(a) the dose comprises a first component for immediate release and a second component for sustained release; and
(b) the weight ratio W of the first component to the sum of the first and second components is less than about 0.25.

69. The method of claim 68 where D is about 20 mg and W is about 0.05.

70. A method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
wherein:
(a) the dosage form provides a mean, single dose, plasma concentration profile that increases substantially monotonically over 24 hours or more;
(b) the dosage form provides a mean, single dose, area under a plasma concentration-time curve for 0-48 hours AUC₀₋₄₈ which satisfies the relationship:
7.6×10⁻³ hour/liter≦AUC₀₋₄₈/D≦16.7×10⁻³ hour/liter; and
(c) the dosage form provides pain relief for about 24 hours or more after administration to the patient.

71. The method of claim 70 wherein AUC₀₋₄₈ and the mean, single dose, plasma concentration profile are determined using plasma samples from individuals to whom one or more opioid antagonists have been administered.

72. The method of claim 70 wherein AUC₀₋₄₈ and the mean, single dose, plasma concentration profile are determined using plasma samples from individuals to whom naltrexone has been administered.

73. The method of claim 70 wherein AUC₀₋₄₈ and the mean, single dose, plasma concentration profile are determined using plasma samples from individuals who have not been administered an opioid antagonist.

74. The method of claim 70 wherein AUC₀₋₄₈ and the mean, single dose, plasma concentration profile are determined using plasma samples from individuals who have not been administered naltrexone.

75. The method of claim 70, 71, or 73 wherein the mean, single dose, plasma concentration profile comprises a first rising phase and a second phase, where the slope of the first rising phase is greater than the magnitude of the slope of the second phase.

76. The method of claim 75 wherein the transition between the first rising phase and the second phase occurs between 12 and 16 hours.

77. The method of claim 76 wherein the first rising phase comprises a first subphase and a second subphase, where the first subphase rises faster than the second subphase.

78. The method of claim 77 wherein the transition between the first subphase and the second subphase occurs between 1 and 3 hours.

79. The method of claim 70, 71, or 73 wherein:
(a) the dose comprises a first component for immediate release and a second component for sustained release; and
(b) the weight ratio W of the first component to the sum of the first and second components is less than about 0.25.

80. The method of claim 79 where D is about 20 mg and W is about 0.05.

81. A method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said dosage form providing (a) a mean, single dose, 12 hour plasma concentration C₁₂ and (b) a mean, single dose, area under a plasma concentration-time curve for 0-48 hours AUC₀₄₈ which satisfy the relationships:
2.7×10⁻⁴ liter⁻¹≦C₁₂/D≦5.7×10⁻⁴ liter⁻¹, and
7.6×10⁻³ hour/liter≦AUC₀₋₄₈/D≦16.7×10⁻³ hour/liter,
wherein said dosage form provides pain relief for about 24 hours or more after administration to the patient.

82. The method of claim 81 wherein C₁₂ and AUC₀₋₄₈ are determined using plasma samples from individuals to whom one or more opioid antagonists have been administered.

83. The method of claim 81 wherein C₁₂ and AUC₀₋₄₈ are determined using plasma samples from individuals to whom naltrexone has been administered.

84. The method of claim 81 wherein C₁₂ and AUC₀₋₄₈ are determined using plasma samples from individuals who have not been administered an opioid antagonist.

85. The method of claim 81 wherein C₁₂ and AUC₀₋₄₈ are determined using plasma samples from individuals who have not been administered naltrexone.

86. The method of claim 81, 82, or 84 wherein:
(a) the dose comprises a first component for immediate release and a second component for sustained release; and
(b) the weight ratio W of the first component to the sum of the first and second components is less than about 0.25.

87. The method of claim 86 where D is about 20 mg and W is about 0.05.

88. A method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone, said dosage form providing mean, steady state, areas under a plasma concentration-time curve for 0-6 hours AUC₀₋₆, 6-12 hours AUC₆₋₁₂, 12-18 hours AUC₁₂₋₁₈, 18-24 hours AUC₁₈₋₂₄, and 0-24 hours AUC₀₋₂₄ which satisfy the relationships:
AUC₀₋₆/AUC₀₋₂₄>0.18,
AUC₆₋₁₂/AUC₀₋₂₄>0.18,
AUC₁₂₋₁₈/AUC₀₋₂₄>0.18, and
AUC₁₈₋₂₄/AUC₀₋₂₄>0.18,
wherein said dosage form provides pain relief for about 24 hours or more after administration to the patient.

89. The method of claim 88 wherein AUC₀₋₆, AUC₆₋₁₂, AUC₁₂₋₁₈, AUC₁₈₋₂₄and AUC₀₋₂₄ are determined using plasma samples from individuals to whom one or more opioid antagonists have been administered.

90. The method of claim 88 wherein AUC₀₋₆, AUC₆₋₁₂, AUC₁₂₋₁₈, AUC₁₈₋₂₄, and AUC₀₋₂₄ are determined using plasma samples from individuals to whom naltrexone has been administered.

91. The method of claim 88 wherein AUC₀₋₆, AUC₆₋₁₂, AUC₁₂₋₁₈, AUC₁₈₋₂₄, and AUC₀₋₂₄ are determined using plasma samples from individuals who have not been administered an opioid antagonist.

92. The method of claim 88 wherein AUC₀₋₆, AUC₆₋₁₂, AUC₁₂₋₁₈, AUC₁₈₋₂₄, and AUC₀₋₂₄ are determined using plasma samples from individuals who have not been administered naltrexone.

93. The method of claim 88, 89, or 91 wherein AUC₀₋₆, AUC₆₋₁₂, AUC₁₂₋₁₈, AUC₁₈₋₂₄, and AUC₀₋₂₄ satisfy the relationships:
AUC₀₋₆/AUC₀₋₂₄>0.20,
AUC₆₋₁₂/AUC₀₋₂₄>0.20,
AUC₁₂₋₁₈/AUC₀₋₂₄>0.20, and
AUC₁₈₋₂₄/AUC₀₋₂₄>0.20.

94. The method of claim 88, 89, or 91 wherein the magnitude of the difference between any two of AUC₀₋₆/AUC₀₋₂₄, AUC₆₋₁₂/AUC₀₋₂₄, AUC₁₂₋₁₈/AUC₀₋₂₄, and AUC₁₈₋₂₄/AUC₀₋₂₄ is less than or equal to 0.05.

95. The method of claim 94 wherein the magnitude of the difference between each of:
AUC₀₋₆/AUC₀₋₂₄ and AUC₆₋₁₂/AUC₀₋₂₄,
AUC₆₋₁₂/AUC₀₋₂₄ and AUC₁₂₋₁₈/AUC₀₋₂₄,
AUC₁₂₋₁₈/AUC₀₋₂₄ and AUC₁₈₋₂₄/AUC₀₋₂₄, and
AUC₁₈₋₂₄/AUC₀₋₂₄ and AUC₀₋₆/AUC₀₋₂₄
is less than or equal to 0.03.

96. The method of claim 88, 89, or 91 wherein the magnitude of the difference between each of:
AUC₀₋₆/AUC₀₋₂₄ and AUC₆₋₂/AUC₀₋₂₄,
AUC₆₋₁₂/AUC₀₋₂₄ and AUC₁₂₋₁₈/AUC₀₋₂₄,
AUC₁₂₋₁₈/AUC₀₋₂₄ and AUC₁₈₋₂₄/AUC₀₋₂₄, and
AUC₁₈₋₂₄/AUC₀₋₂₄ and AUC₀₋₆/AUC₀₋₂₄
is less than or equal to 0.03.

97. The method of claim 88, 89, or 91 wherein:
(a) the dose comprises a first component for immediate release and a second component for sustained release; and
(b) the weight ratio W of the first component to the sum of the first and second components is less than about 0.25.

98. The method of claim 97 where D is about 20 mg and W is about 0.05.

99. A method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
said dosage form providing pain relief for about 24 hours or more after administration to the patient and having an in vitro release profile in which:
(a) 0-20% of the dose is released in 0-2 hours;
(b) 30-65% of the dose is released in 0-12 hours; and
(c) 80-100% of the dose is released in 0-24 hours;
where the release profile is determined using a USP Type VII bath indexer in a constant temperature water bath at 37° C.

100. The method of claim 99 wherein 33-63% of the dose is released in 0-12 hours.

101. The method of claim 99 wherein:
(a) the dose comprises a first component for immediate release and a second component for sustained release; and
(b) the weight ratio W of the first component to the sum of the first and second components is less than about 0.25.

102. The method of claim 101 where D is about 20 mg and W is about 0.05.

103. A method of treating pain in humans comprising orally administering to a human patient on a once-a-day basis a controlled-release dosage form comprising a dose D of:
(i) oxycodone,
(ii) one or more pharmaceutically-acceptable acid addition salts of oxycodone, or
(iii) a combination of oxycodone and one or more pharmaceutically-acceptable acid addition salts of oxycodone,
wherein:
(a) the dose comprises a first component for immediate release and a second component for sustained release;
(b) the weight ratio W of the first component to the sum of the first and second components is less than about 0.25; and
(c) the dosage form provides pain relief for about 24 hours or more after administration to the patient.

104. The method of claim 103 wherein W is less than about 0.10.

105. The method of claim 103 wherein W is less than or equal to about 0.05.

106. The method of claim 103 where D is about 20 mg and W is about 0.05.
